(19) United States
(12) Patent Application Publication
(10) Pub. No.: US 2021/0263018 A1
(43) Pub. Date: Aug. 26, 2021

(54) LATERAL FLOW TEST STRIP IMMUNOASSAY IN VITRO DIAGNOSTIC DEVICE

(71) Applicant: EXPERIMENT X LTD., London (GB)

(72) Inventor: Alexander TARAN, London (GB)

(21) Appl. No.: 17/261,623

(22) PCT Filed: Jul. 22, 2019
(86) PCT No.: PCT/GB2019/052050

§ 371 Date: Jan. 20, 2021

(30) Foreign Application Priority Data
Jul. 20, 2018 (GB) 1811927.1

(51) Int. Cl. G01N 33/53; G01N 21/78; G01N 21/64; H04N 9/04; G06T 5/00; H04N 5/225; G06T 7/80; H04N 5/247; H04N 9/64; H04N 17/00; G06T 7/00; G06T 7/90; G06K 19/06; G06K 19/07

(52) U.S. Cl. CPC G01N 33/5302; G01N 2021/7759; G01N 21/6428; H04N 9/04517; G06T 5/006; H04N 5/2256; G06T 7/80; H04N 5/247; H04N 9/646; H04N 17/002; G06T 7/0016; G06T 7/90; G06K 19/06037; G06K 19/0723; G01N 21/78

(57) ABSTRACT

A portable, rechargeable lateral flow test strip immunoassay in vitro diagnostic device includes a color imaging sensor for imaging a lateral flow test strip, a lens that images a substantial part of the 2D surface of the lateral flow test strip onto the color imaging sensor and image processing software that identifies patterns in the test lines and/or dots or other distributions in the test strip and compensates for distortions, non-uniformities or anomalies in the lines and/or dots or other distributions in the test strip.

### BACKGROUND OF THE INVENTION

### 1. Field of the Invention

The field of the invention relates to a portable, rechargeable lateral flow test strip immunoassay in vitro diagnostic device.

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### 2. Description Prior Art

Existing LF based tests detect the presence (or absence) of a target analyte in a sample. The sample can be: whole blood, capillary blood, serum, plasma, urine, saliva, feces which may be used alone or may be mixed/followed by the buffer specific to the test. They allow for the detection and measurement of a variety of biomarkers, pathogens, mycotoxins, cells, nucleic acid detection and are used in medical, consumer, food, agriculture, environmental and veterinary testing.

LF-based tests are widely used for medical diagnostics either for home testing, point of care testing, laboratory or hospital use. A well-known application is the home pregnancy test.

There is no device available on the market able to perform the read-out of different types of rapid tests such as: electrochemical/amperometric, fluorescent, photometric or enzymatic immunochromatographic rapid tests.

Imaging processing algorithms are often used to increase the accuracy of the interpretation of the test results. However, current imaging processing algorithms are often specifically designed for only a particular test type and medium.

Tests results can be interpreted qualitatively or quantitatively. Qualitative tests do not require any calibration process, as the interpretation is limited to just a “yes”/“no” result.

For quantitative tests, calibration information is supplied together with the test kit in the form of an SD card or it is encoded into a QR or bar-code. However a disadvantage of many commercially LF systems is that the calibration data is formed just once after an initial batch calibration is performed and it is not recalculated or revised after one or more test results has been obtained.

Further a lens may cause image distortion and may contain aberrations, that can decrease the accuracy of a test result. However, the imaging processing algorithms that are being used in today's diagnostic devices do not take this into account.

There is a need for a low cost, small and easy to use portable diagnostic device that would work for any available type of tests and that would require minimal user, intervention, while at the same time giving fast and accurate results.

The present invention addresses the above vulnerabilities and also other problems not described above.

### SUMMARY OF THE INVENTION

There are fourteen aspects to the invention. We list these as aspects A-N below. Further details are at Appendix 1.

A. Software Compensation for Lens Distortions

A portable, rechargeable lateral flow test strip immunoassay in vitro diagnostic device, including a colour imaging sensor for imaging a lateral flow test strip, including a lens that images a substantial part of the 2D surface of the lateral flow test strip onto the colour imaging sensor and image processing software that identifies patterns in the test lines and/or dots or other distributions in the test strip and compensates for distortions, non-uniformities or anomalies in the lines and/or dots or other distributions in the test strip.

B. Imaging Adjacent Test Strips

A portable, rechargeable lateral flow test strip immunoassay in vitro diagnostic device, including a colour imaging sensor for imaging the lateral flow test strip, including a lens that simultaneously or near simultaneously images a substantial part of the 2D surface of two or more adjacent immunoassay test strips.

C. Using a Wide Angle Lens

A portable, rechargeable lateral flow test strip immunoassay in vitro diagnostic device, including a colour imaging sensor for imaging the test strip, including a wide angle lens that images a substantial part of the 2D surface of the immunoassay test strip onto the colour imaging sensor.

D. Using Several Camera Modules

A portable, rechargeable lateral flow test strip immunoassay in vitro diagnostic device, including two or more camera modules, each camera module including an imaging sensor and a lens that images a part of the 2D surface of the immunoassay test strip onto the imaging sensor, and in which the two or more camera modules have different specifications.

E. Automatic Self-Calibration Using Colour and/or White Charts Built Into Part of the Device and on a Dedicated Cartridge.

A portable, rechargeable lateral flow test strip immunoassay in vitro diagnostic device, including a colour imaging sensor for imaging the test strip, including a wide angle lens that images a substantial part of the 2D surface of the test strip onto the colour imaging sensor and that is configured to automatically self-calibrate in the colour space by imaging a colour and/or white chart, in which the colour and/or white charts are built into part of the device and are also present on a dedicated cartridge.

F. Analysing Lateral Flow Strips and Also Micro-Arrays

A portable, rechargeable lateral flow immunoassay test strip in vitro diagnostic device, including a colour imaging sensor for imaging the test strip, including a lens that images a substantial part of the 2D surface of the immunoassay test strip onto the colour imaging sensor and that is configured to receive and also analyse cartridges for both lateral flow immunoassay test strips and also micro-array immunoassays.

G. Illuminating with a UV Light Source

A portable, rechargeable lateral flow immunoassay test strip in vitro diagnostic device, including a colour imaging sensor or black and white imaging sensor for imaging the test strip, including a wide angle lens that images a substantial part of the 2D surface of the immunoassay test strip onto the colour imaging sensor and that includes a UV light source to illuminate the immunoassay test strip and.

H. Cloud-Connectivity via Bluetooth to a Smartphone

A portable, rechargeable lateral flow immunoassay test strip in vitro diagnostic device, including a short range, secure communications link or interface that enables the device to exchange data with an application running on a smartphone or other wireless device, and the smartphone or other wireless connected device then connects over a secure link via the internet or a cellular network to a remote, cloud-based server.

I. Cartridge Stores the Kinetic Data and the Calibration Curve

A cartridge for a portable, rechargeable lateral flow immunoassay test strip in vitro diagnostic device, the cartridge including or associated with a read/write memory to which is written, in normal operation by the device, the kinetic data (i.e. data from a kinetic analysis) and the calibration curve obtained when the device images a test strip mounted on or associated with that cartridge.

J. Cartridge Accepts a Lateral Flow Test Strip and Also a Micro-Array Test Strip

A cartridge for a portable, rechargeable lateral flow immunoassay test strip in vitro diagnostic device, the cartridge configured to receive either a lateral flow test strip or a micro-array test strip.

K. Cartridge is Locked with a Crypto-Chip to Ensure Authenticity

A cartridge for a portable, rechargeable lateral flow immunoassay test strip in vitro diagnostic device, the cartridge including a secure memory chip or device and a communications chip or device, the secure memory storing a unique identification code or crypto-key or number and the cartridge configured to undertake a handshake or other protocol in which the unique identification code or crypto-key or number is checked and verified as authentic or otherwise genuine.

L. LEDs are Switched On or Off Selectively in Order to Improve Accuracy of the Results

A portable, rechargeable lateral flow immunoassay test strip in vitro diagnostic device, including a colour imaging sensor for imaging the test strip, including a lens that images a substantial part of the 2D surface of the immunoassay test strip onto the colour imaging sensor and that includes multiple LEDs of different colours used to read the test strip, in which the device can be programmed to select only a set of LEDs to be turned on, in order to image a specific region of interest of the 2D surface of the immunoassay test strip onto the colour imaging sensor.

M. Kinetic Analysis of the Test Strip to Provide Faster Results

A portable, rechargeable lateral flow test strip immunoassay in vitro diagnostic device, including a colour imaging sensor for imaging a lateral flow test strip, including a lens that images a substantial part of the 2D surface of the lateral flow test strip onto the colour imaging sensor, and in which the device is configured to capture the progress or change or rate of change of the lateral flow test strip as a function of time (‘kinetic data’).

N. Simplified User Interaction with the Device

A portable, rechargeable lateral flow test strip immunoassay in vitro diagnostic device, including a colour imaging sensor for imaging the lateral flow test strip, including a lens that images a substantial part of the 2D surface of the immunoassay test strip onto the colour imaging sensor and that is configured to automatically run a diagnostic result following a one-step user interaction with the device.

### BRIEF DESCRIPTION OF THE FIGURES

Aspects of the invention will now be described, by way of example(s), with reference to the following Figures, which each show features of the invention:

FIG. 1 shows a perspective view of a portable rechargeable lateral flow test strip immunoassay in vitro diagnostic device customized test cassettes adaptor

FIG. 2 shows the aperture adaptor which can be inserted inside the aperture of the diagnostic device.

FIG. 3 shows a bottom perspective view of the diagnostic device.

FIG. 4 shows a cross section of the diagnostic device.

FIG. 5 shows another portable rechargeable lateral flow test strip immunoassay in vitro diagnostic device with mechanical or touch-sensitive power button.

FIG. 6 shows a portable rechargeable lateral flow test strip immunoassay in vitro diagnostic device with commercially available test cassette set into a customized adaptor.

FIG. 7 shows an exploded view of a multiple strips test set into a customized adaptor.

FIG. 8 shows the inside components of the diagnostic device.

FIG. 9 shows the steps performed during a calibration procedure.

FIG. 10 shows the camera field of view when the dedicated calibration cassette is inserted inside the LF reader

FIG. 11 shows the image sensor module reading out the “reference zones” or colour charts.

FIG. 12 shows the image sensor module reading out a lateral flow test strip.

FIG. 13 shows the image sensor module reading out multiple lateral flow test strips.

FIG. 14 shows the image sensor module reading out a microarray lateral flow test strip.

FIG. 15 shows the image sensor module reading out a microarray lateral flow test strip set into the customized adaptor.

FIG. 16 shows a block diagram illustrating the steps performed while reading out multiple lateral flow test strips set into one test cassette.

FIG. 17 shows a flow diagram summarizing the distortion compensation algorithm.

FIG. 18 shows a comparison of results obtained with LF test readers without colour charts and with colour charts.

FIG. 19 shows a flow diagram illustrating key steps of the image correction algorithm.

FIG. 20 shows a plot of the image density of a test strip.

FIG. 21 shows an example of a matched filter

FIG. 22 shows a plot of the convolution of the image density with different matched filter.

FIG. 23 shows a diagram of the image sensor module.

FIG. 24 shows a diagram with another configuration for the image sensor module.

FIG. 25 shows a camera module imaging a cartridge comprising test strips.

FIG. 26 shows an illustration of switching on LEDS on a cartridge.

FIG. 27 shows a top view of a test strip with image glares resulting from the illumination.

FIG. 28 shows an illustration of switching on LEDs in a sequence on a cartridge.

FIG. 29 shows a cartridge with two test strips with image glares and shades.

FIG. 30 shows the result of combining images illuminated with different sequence of LEDs.

FIG. 31 shows a plot with kinetic results performed on a pregnancy test strip.

FIG. 32 shows the progress of a test line on a cartridge at a first time and at a second time

FIG. 33 shows a photometric test inserted in a LF test reader and an electrochemical test inserted in a LF test reader.

FIG. 34 shows the cross section of a cartridge carrying adaptor for photometric tests and for electrochemical test.

FIG. 35 shows a portable diagnostic device in communication with an application on a connected device.

FIG. 36 shows an example of a screenshot of a mobile application.

FIG. 37 shows an example of a screenshot of a mobile application.

FIG. 38 shows an example of a screenshot of a mobile application.

### DETAILED DESCRIPTION

The portable rechargeable lateral flow test strip immunoassay in vitro diagnostic device described below is intended to be used in hospitals, clinics, doctors' offices, in-the-field or at home facilities, for remote control and monitoring of one or multiple health conditions.

The compatibility of the diagnostic device with EHR (Electronic Health Record), LIS (Laboratory Information System) and HIS (Health Information System) provides possibilities for integration with diagnostic medical databases and systems to enrich medical records of patients with tests results data in an automatic manner.

1. Overview of the System

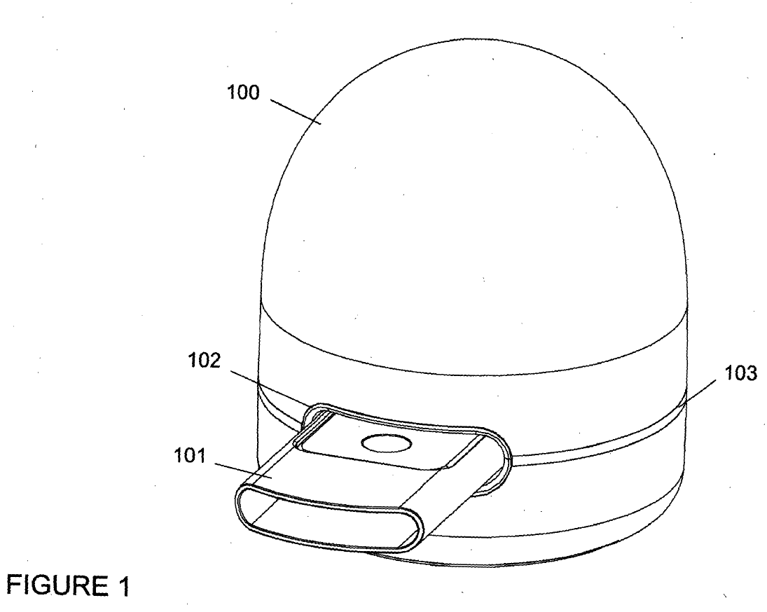

FIG. 1 shows a portable rechargeable lateral flow test strip immunoassay in vitro diagnostic device (100) or lateral flow (LF) test reader. The device is relatively small. (palm/hand size format) and lightweight, with a weight of approximately 270 g and may, with material and design improvement weigh less than 270 g. The device allows for reading and testing of a lateral flow test strip held in a cartridge (101). The cartridge (101) is configured by shape and size to be partly inserted into a removable aperture (102) of the device (100).

The diagnostic device includes an LED ring (103) extending around the circumference the reader's body that provides a visual indicator to indicate the status of the LF test reader and/or the status of the cartridge. The visual indicator changes appearance as for example a cartridge is being read, or when the diagnostic process is finished, or when an error occurs.

The visual indicator may provide visual feedback in a number of ways, such as different colours, flashing or blinking patterns. In addition, the visual indicator may also be combined with an audio indicator, tactile indicator or a vibration indicator. As an example, the following status are detected by the reader and are automatically provided to the user with the following, but not limited to:
Test in progress: green moving ring;
Hardware error: red & yellow, blinking ring;
Inappropriate environmental conditions: red, blinking ring;
Inappropriate reader inclination angle;
Battery level is less 10% red, blinking ring;
Battery level from green to yellow;
Idle mode:
   IDLE, battery charger is disconnected: green, constant;
   IDLE, charging battery: yellow, blinking ring;
   Firmware update is in progress: blue moving ring;
   Device firmware (ready for FW update): blue;
Operational status:
   application running on a connected device is connected: blue, blink for 2 seconds;
   IDLE, charging is in progress (green, yellow), constant ring;
   Assay processing is failed due to cartridge absence: yellow, blinking ring;
   Assay processing is in progress: green, moving ring;
   Assay processing is completed: same as “Idle”;
   Assay processing has failed due to invalid cartridge: yellow, blinking ring;
   Ready for pairing via BLE (Bluetooth Low Energy) & BLE pairing status;
   Ready for WiFi network configuration & WiFi;
   Connectivity status;
Interaction with Igloo:
   Insert cartridge (simultaneously with UI message on an application running on a connected device): green light from the middle to the cartridge window;
   Eject cartridge (simultaneously with UI message on an application running on a connected device): green light from the cartridge window to the middle.

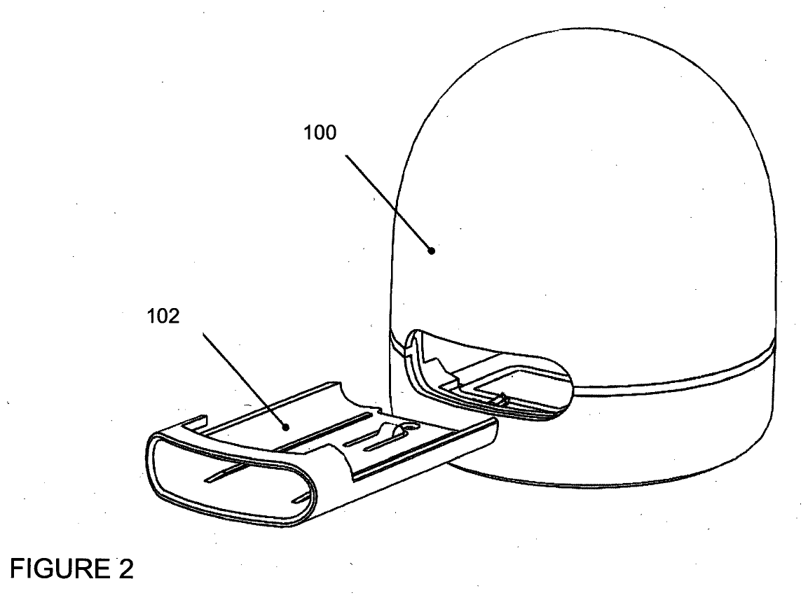

FIG. 2 shows a removable aperture (102) which can be inserted inside the aperture of the LF test reader (100). The removable aperture (102) is available in different sizes and therefore allows for different sizes of cartridge to be inserted inside the aperture of the LF test reader.

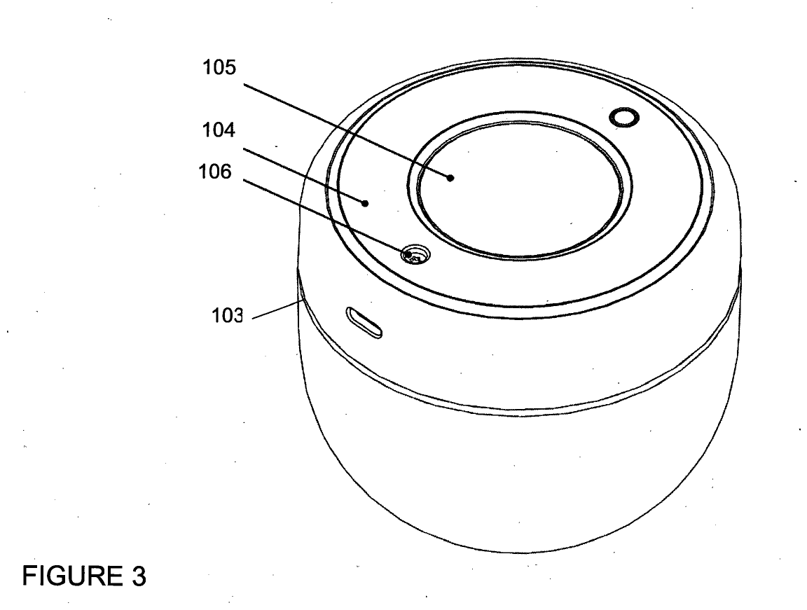

The removable aperture (102) is easily attached to the LF test reader via a screw (106) accessible underneath the LF test reader as seen in FIG. 3. The aperture can therefore be easily inserted, attached or removed without having to open the LF test reader. The bottom of the device may also include an antislider carpet (104).

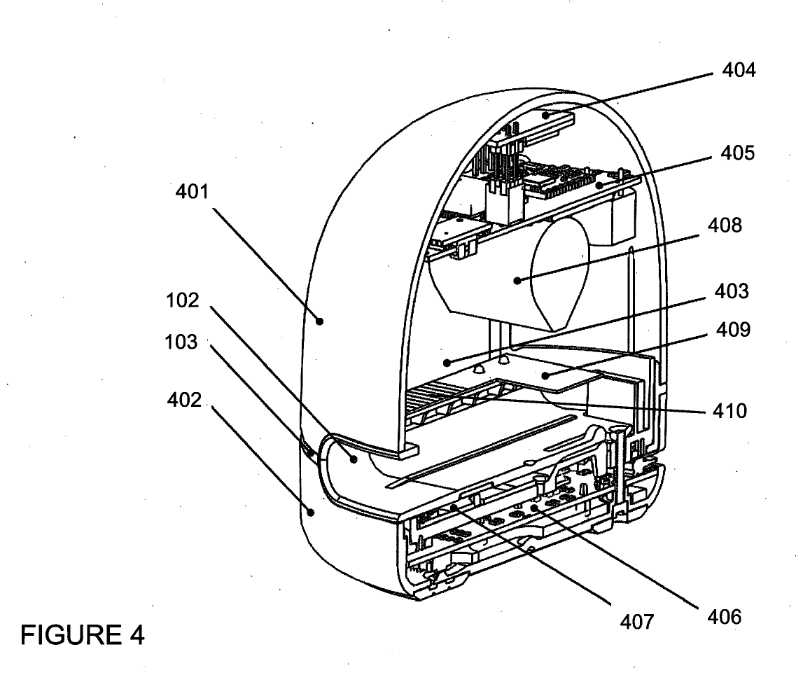

FIG. 4 shows a cross section of the LF test reader including a top section (401) and a bottom section (402). The key internal components include: an internal bearing component (403), a touch-pad panel (to switch the device on or off and to pair the device) (404), a pcb (405) with a camera module including an image sensor and lens, a plurality of LEDs, a pcb (407) including a NFC antenna with a wireless charging function, an accumulator (408) and a plate (409) including “reference zones” (410). The reference zones or colour and/or white charts are located inside the LF test reader on both sides of the slider that is used to load the cartridge or cartridge carrying adaptor.

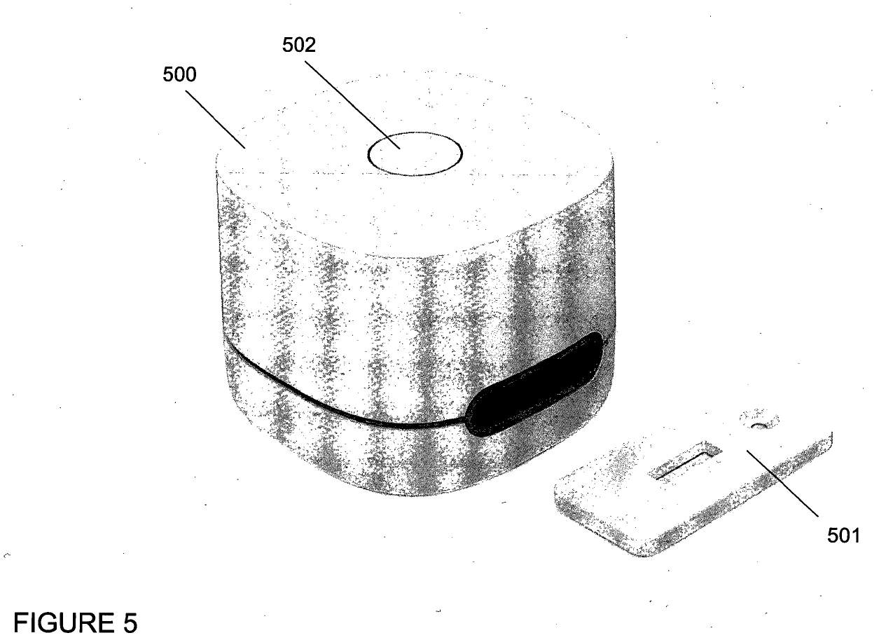

With reference to FIG. 5, an alternative design for the portable rechargeable lateral flow test strip immunoassay in vitro diagnostic device (500) is shown. The picture shows the device (500) alongside a test strip held in a cartridge (501). In this example, the visual indicator is a LED ring (502) around a power button placed on top of the diagnostic device.

Preferably, the diagnostic device is automatically switched on when the cartridge or cartridge carrying adaptor is slid onto the diagnostic device. This allows the device to be used more easily and requires minimal user intervention.

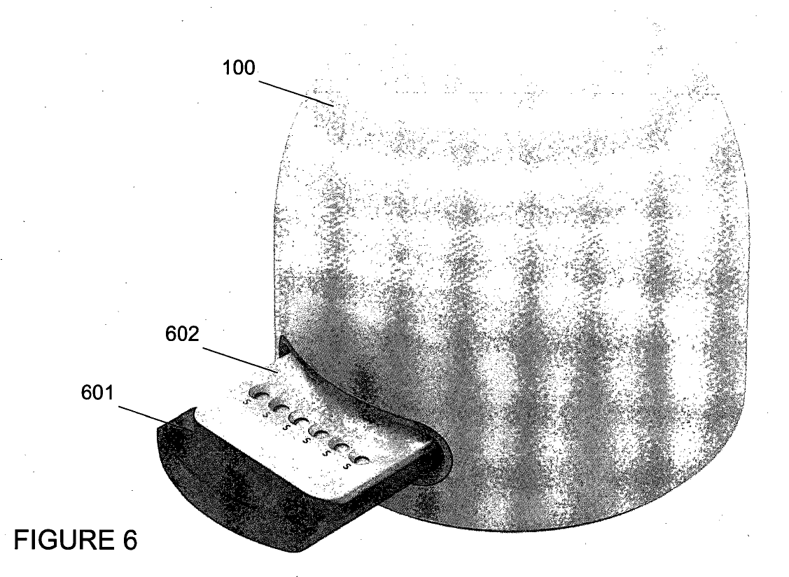

Alternatively, a cartridge carrying adaptor (601) may be used to hold the cartridge (602) as shown in FIG. 6. The cartridge carrying adaptor is also configured by shape and size to slide into the aperture of the device (100). The cartridge can receive any lateral flow immunoassay test strips and any micro-array immunoassays.

The device therefore operates with any rapid lateral flow assays, test strips, dipsticks test or any other tests that are commercially available.

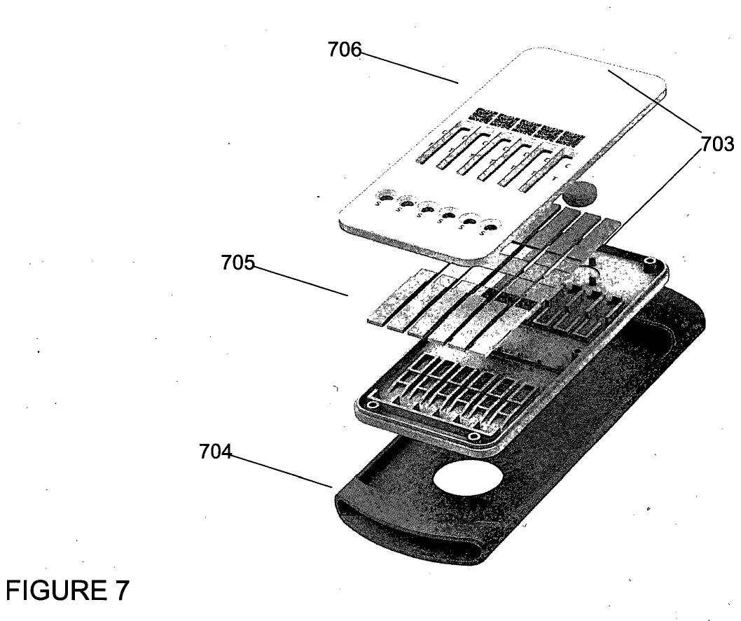

FIG. 7 shows an exploded view of a cartridge (703) held inside a cartridge carrying adaptor (704). The cartridge contains one or more test strips (705). In this example, the cartridge has been configured to receive 6 test strips. Each test strip is associated with a QR code (706) that is engraved or printed on the cartridge.

Alternatively, an NFC, or RFID tag or other memory may be included on the cartridge or cartridge carrying adaptor. The QR code or NFC or RFID tag or other memory holds data related to a whole batch of test strips. The data includes one or more of the following: batch or lot number, test number, batch or test type, exposition time, time of the test, calibration data, kinetic data, (see later), test manufacturer, test manufacturer address, test manufacturer contact information, expiration date, calibration curve for this batch etc.

As each cartridge adaptor holds data related to a specific test, batch or lot number, they are dedicated to be used with a specific cartridge or test strip. The test type of the cartridge is then automatically detected by reading the data from the NFC or QR code.

The cartridge also includes a secure memory chip that is used to store a unique identification code or crypto-key or number. The cartridge is then configured to undertake a handshake or other protocol in which the unique identification code or crypto-key or number is checked and verified as authentic or otherwise genuine.

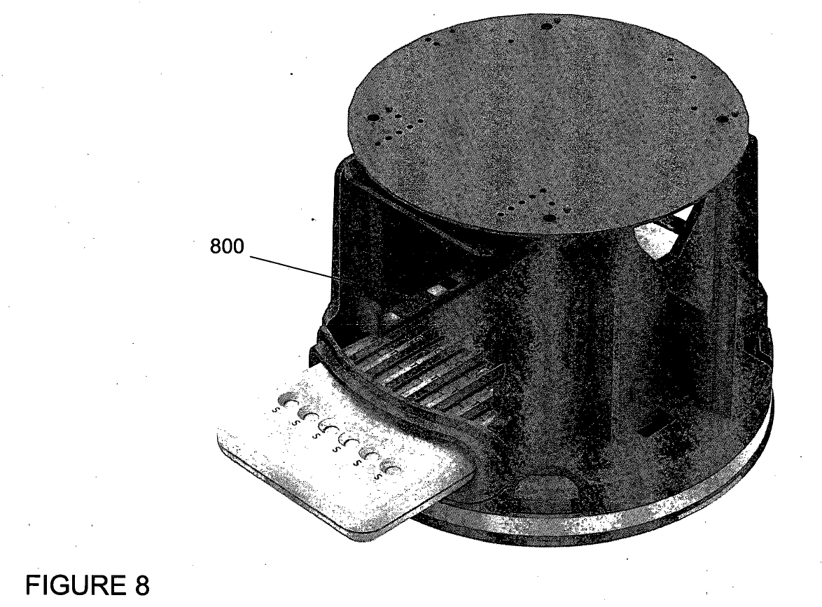

FIG. 8 shows the inside components of the portable rechargeable lateral flow test strip immunoassay in vitro diagnostic device. A sample (e.g. whole blood, serum, urine, saliva, feces and plasma) is first inserted into the cartridge, and the cartridge or cartridge carrying adaptor is loaded into the diagnostic device (100). The diagnostic device includes an RFID module that reads any batch and test information that is held in one or more QR code, or RFID tag or memory on the cartridge or cartridge carrying adaptor. The internal components of the device include “reference zones” or colour and/or white charts (800).

The diagnostic device further includes an imaging sensor that captures a high resolution image of the one or more test strips, and an imaging processing software that processes the captured image to provide high accuracy test results. If more than one test strip is present in the cartridge, the diagnostic device sequentially, near simultaneously or simultaneously images the test strips together. The diagnostic device is able to sequentially, near simultaneously or simultaneously read-out up to at least 6 standard test strips. Further, the diagnostic device is able to read out multiple test lines on the same test strip. For example, 8 lines may be read on the same test strip.

Additionally, the LF test reader may include a UV light source to illuminate the immunoassay test strip, and a UV filter positioned in front of the colour or black and white imaging sensor. This configuration will result in an increase in the sensitivity and limit of detection in fluorescent-based lateral flow tests, in which for example antibodies may be labeled with fluorescent dyes or europium particles.

Existing CMOS camera-based devices intended for lateral flow test performance usually have a good illumination but not a wide FOV, which does not allow to perform the read out of several test strips and QR codes or any other additional info from the test cassette.

Smartphone based readers have a wider FOV however they suffer from higher lighting unevenness, glares and shades problems, often resulting in the distortion of test results read-out.

Several cameras inside a single reader may be used in order to increase a camera's FOV. The LF test reader will remain low cost thanks to the relatively low BOM costs of commercial cameras.

Moreover, cameras with different specifications can also be included in a single device. Different specifications may include: different FOVs, different filters or different sensitivities. As compared to using a single camera, the device's production cost will increase insignificantly.

For example, a wide-angle camera can be used for capturing a general view of the test cassette (QR code and different inscriptions on test cartridge) and a narrow-angle camera can be used for accurate and precise microarray read-out.

As another example, a further combination of two or more cameras may be used in which a first camera for capturing a general view of the test cassette may be used in combination with a highly sensitive camera destined for fluorescent rapid tests readout including a black and white matrix with higher pixels size and a narrow viewing angle. The highly sensitive camera collecting more light than the first camera.

As another example, a first camera may be used with a narrowband or UV filter to read out fluorescent-based lateral flow tests and a camera without a UV filter may be used to read out conventional LF-based tests.

A camera including a global shutter may also be combined with a camera including a rolling shutter.

In addition, cameras with infrared pixels for readout of tests changing colour in the infrared ranges may also be used.

Measurement Errors

The accuracy of commercially available rapid tests differs greatly with qualitative, quantitative or semi-quantitative rapid tests being available on the market. Historically LF tests were visually evaluated with the highest practical application being reported with very high coefficient variation (CVₜ): around 25-30%. Lately, the emergence in the numbers of readers in the market of quantitative tests with much lower coefficient of variation of around 3-20% gained popularity.

The coefficient of variation of results read-outs may be defined by the following:

CV=\sqrt{CVₜ²CV_{b}²+CVᵣ²+CVᵣᵣ²+CV_{glare}²},

Where CVₜ is the coefficient of variation from a test, CV_{b} is coefficient variation between test batches, CVᵣ is the coefficient variation from test results obtained from a single reader, CVᵣᵣ is the coefficient variation between readers and CV_{glare} are additional errors due to the reflection from the cartridge. Minimizing the coefficient variation results in an increase of the accuracy, sensitivity and reliability of test results obtained by the LF test reader and also reduces the number of false positive and/or false negative results.

2. Calibration Setup

Devices manufacturers highly recommended to perform a calibration procedure at regular intervals, such as once every several days or once a week or couple of weeks.

As a comparison, many LF test readers use expensive CCD image sensors in order to avoid the device's recalibration as they provide a set dark current and low background noise level.

CCD LEDs linear used in certain devices also require a systematic calibration in the course of operation or use. Readers with moving LEDs also need to be recalibrated from time to time.

CMOS sensors are considerably worse, usually due to the unstable dark current and high noise levels. However, CMOS sensors also provide the most affordable and acceptable off the shelf solution. The purchasing price of the customized camera module is $7 or less, such as $5 or less.

The CMOS sensor may have for example a resolution of about 2 megapixels or less. The CMOS sensor may also have a pixel size of about 1.75 μm.

The location of reference zones or calibrator charts inside the LF test reader allows the device to perform an automatic self-calibration when switched on and therefore allows for the compensation of colour reproduction, temperature alterations, change of the LED's brightness or of any parameters affected by time of use. Therefore the necessity of a re-calibration of the device from an experienced user or manufacturer is reduced.

The following procedures ensure that data from multiple diagnostic devices can be read and analysed consistently regardless of external factors such as ambient temperature or light conditions. Specifically, the imaging software is able to compensate for temperature dependent drift in the colour mapping of the imaging sensor.

Additionally an improvement in the dynamic range of test line intensity is achieved.

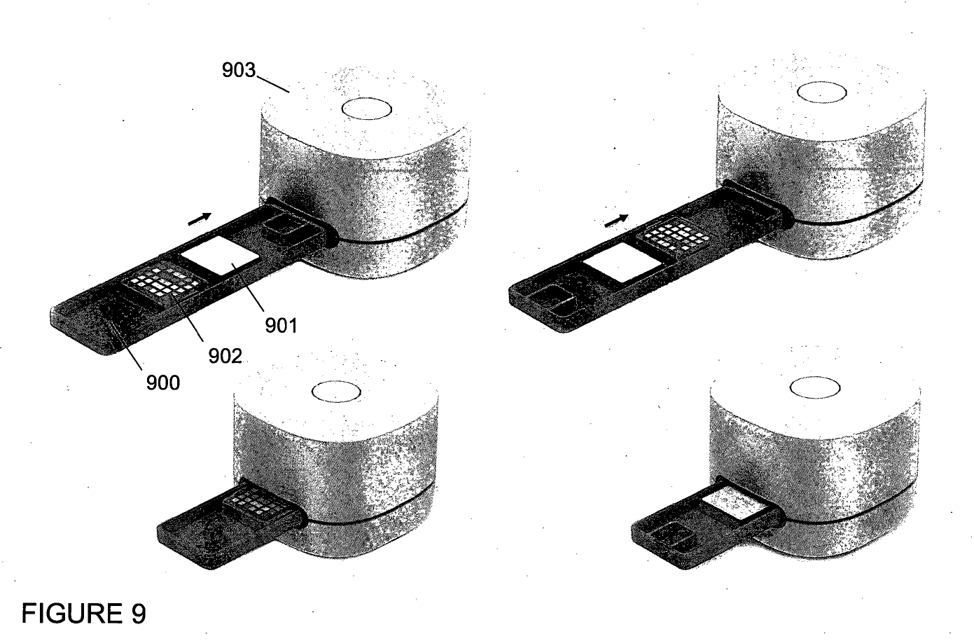

With reference to FIG. 9, a calibration procedure is illustrated. The calibration procedure uses a dedicated calibration cartridge (900) including a white card (901) and a colour matrix card, such as a Macbeth colour chart (902). A two step calibration procedure is performed in which the dedicated calibration cartridge (900) is inserted into the device (903). The device first reads the white card (901) and then the colour matrix card (902). The calibration data is then stored in the device's memory.

Dedicated cartridge is considered as a gold standard and the colors embedded in the device itself are equated to the “gold standard’ and they become a reference gold standard color charts by that mean.

A calibration procedure is as follows:
The results from reading the white card are analysed to determine an optimal exposition value and to calculate calibration parameters for vignette compensation.
The results from reading out the color calibration charts are used for calculating the following calibration parameters: image scale factor, a color correction matrix and the camera's displacement.
The reference zones located inside the LF test reader are automatically detected, a read-out of each reference zones color is performed and the results are stored in the device's memory.

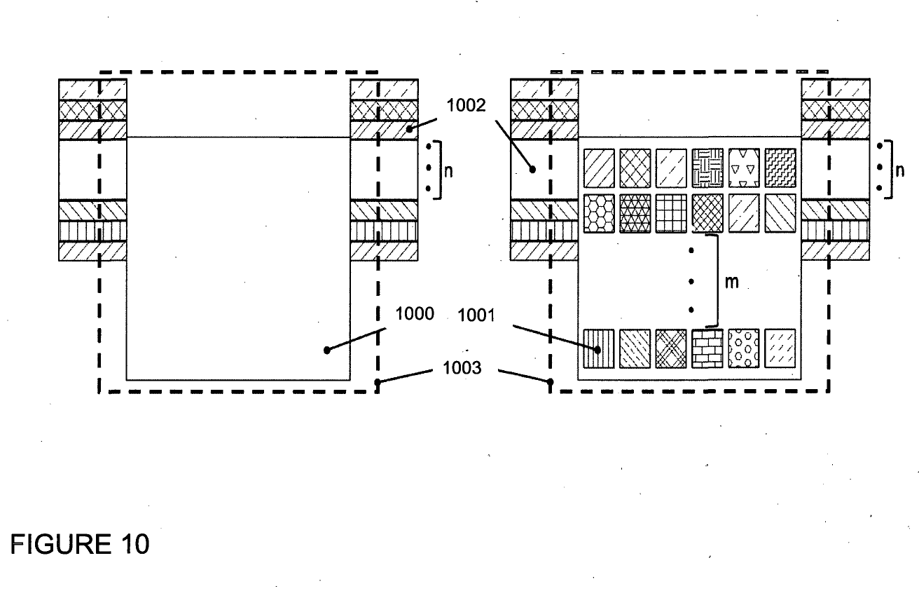

FIG. 10 shows the camera field of view when the dedicated calibration cartridge is inserted inside the LF reader including a white card (1000), a colour matrix card (1001), and reference zones (1002) comprising a number of color charts.

Performed calibration data may also be stored in the cloud for subsequent analysis such as for comparing with reference values.

3. Procedure for Reading Out a Test Strip

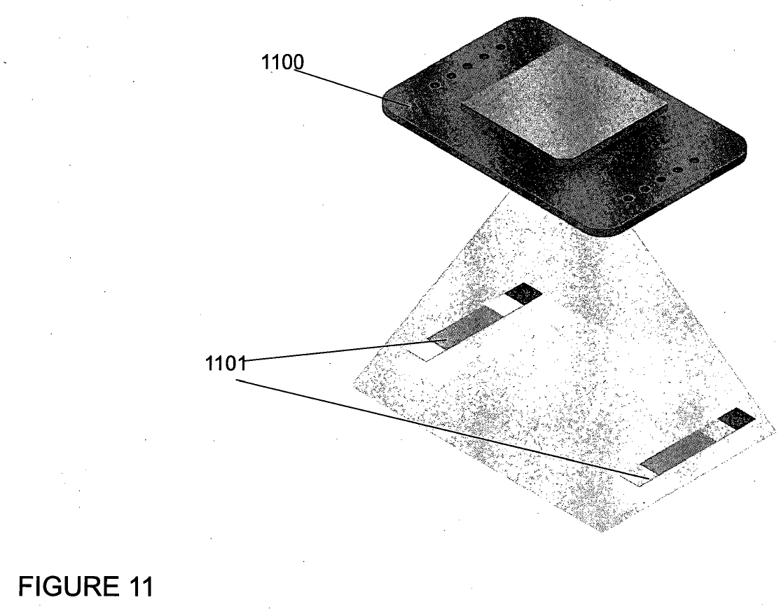

Prior to reading each test strip, the reader automatically initiates a comparison procedure, as shown in FIG. 11. The imaging sensor module (1100) automatically reads out the colour charts or reference zones (1101) and saves the results in the device's memory. The device compares the reference zones results with the stored calibration results, previously stored in the reader's memory, using for example a least squared method of comparison.

The device reads color codes from the reference zones in order to sequentially correct the parameters for each channel (red, green, and blue) independently. In case a difference is revealed, the image is appropriately corrected in accordance with the colors stored in the diagnostic device's memory of “true colors”, obtained from the calibration procedure (as shown in FIG. 9).

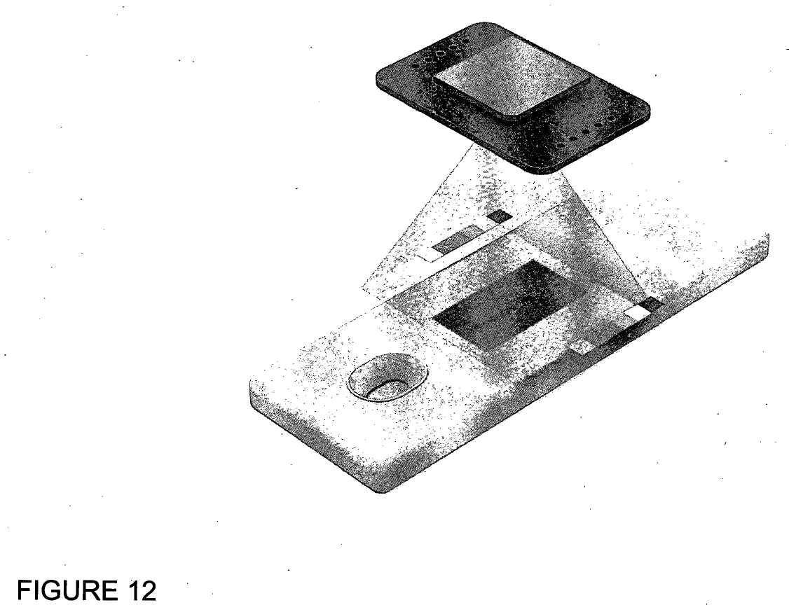

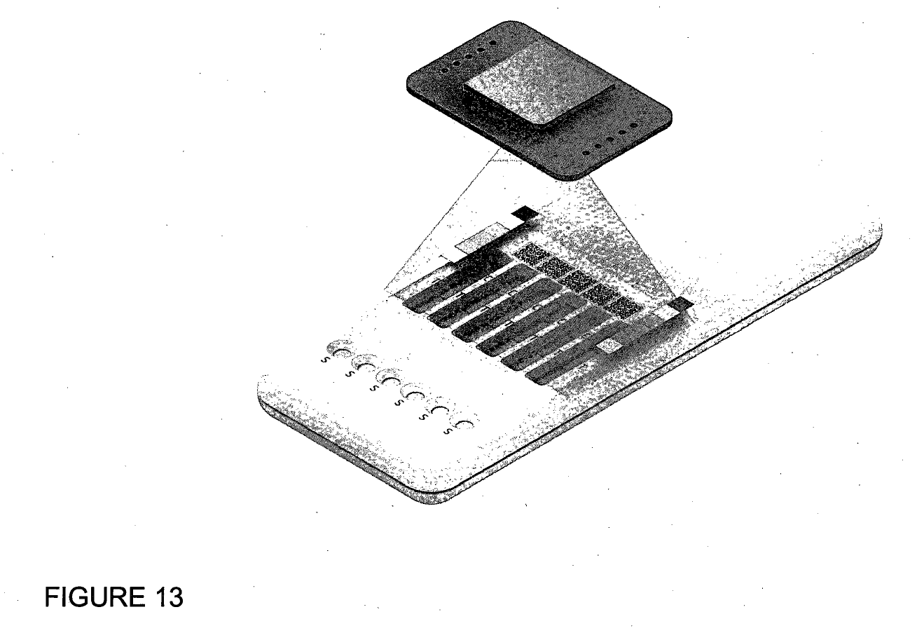

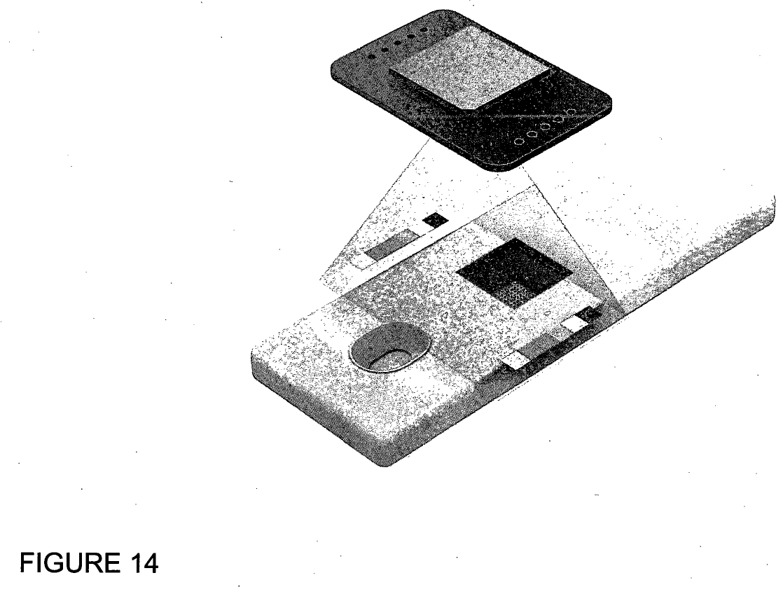

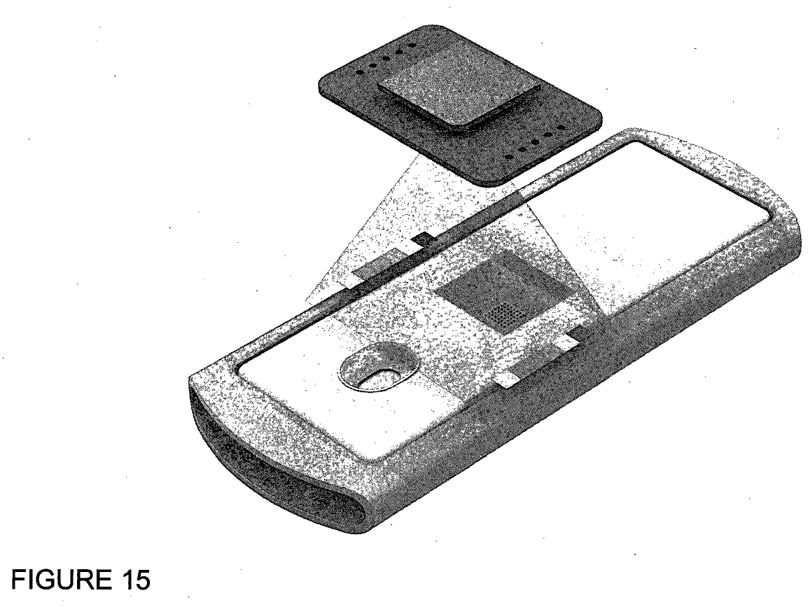

Following the initial calibration and comparison procedures, the device is able to read one test strip (FIG. 12), multiple test strips (FIG. 13) or microarray sequentially, near simultaneously or simultaneously (FIGS. 14 and 15).

The diagnostic device is able to perform about 200 different tests in autonomous mode—without an external power supply and connectivity to a connected application or a cloud platform.

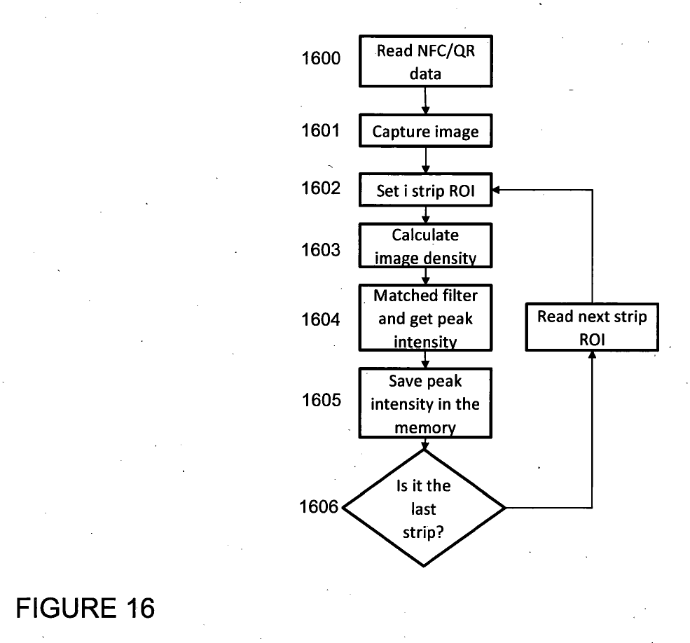

With reference to FIG. 16, a block diagram describes the different steps performed in order to read multiple test strips. At the end of testing procedure preparation, or at a programmed time interval stored on the cartridge memory, the device performs the following steps:
The device reads the NFC or RFID or QR data from the cartridge or cartridge carrying adaptor (1600). At the end of the preparation phase, or at a programmed time interval stored on the cartridge memory:
The device captures an image of the cartridge under test (1601) as described in the following section;
The number of strips or regions of interest (ROI) is set to i (1602);
The devices measures or calculates the intensity of lines or dots of each strip (1603);
A matched filter is applied to the image density and the peak intensity for each line or dot is obtained (1604);
The peak intensity of lines or dots of each strip is determined and saved in the device memory (1605);
The device repeats the procedure until all the strips and ROI have been read (1606).

Additionally, raw data test results are transmitted to a local or external data storage for subsequent analysis of the test results. The analysis of the raw data may also be used to modify calibration data in order to improve the accuracy or sensitivity of subsequent test results.

Storing the test results as well as the calibration data provides a high level of traceability. It can also be used to ensure the compliance, such as General Data Protection Regulation compliance, of the test results performed and to help improve test results or procedures. This may be crucial for example for forensic analysis.

4. Distortion Compensation Algorithm

This section provides a detailed description of a distortion compensation algorithm used by the system. A distortion refers generally to anything that would make an image imperfect and which can ultimately affect the accuracy of a test result.

Each device possesses its own unique distortions of the lens and LEDs. Error or distortion sources may come from the following but are not limited to:
Vignetting effect (in which the brightness intensity at the centre of the image is higher as compared to the periphery);
Non-uniformity of the illumination of the cartridge;
Different exposition time from one camera to another;
Color distortions (Color reproduction of each color channel may be different);
Color variation during operation (LEDs may change their intensity, a camera's photodiode often has strong temperature dependency);
Camera's displacement;
Accuracy of converting pixels to mm (lens may be manufactured from different molds—i.e. FOV (Field of View) can differ considerably).

The distortion compensation algorithm helps in minimising the coefficient variation between readers. The algorithm takes into account the lens parameters, the location of the LEDs in relation to the lens and the specific test parameters, the position of the test strip and of the test and control lines in relation to the cartridge or cartridge-carrying adaptor.

The algorithm further takes into account the LEDs configuration, such as the sequence of color used for the LEDs, as well as the calibration results from reading out the white card and colour matrix card as described above.

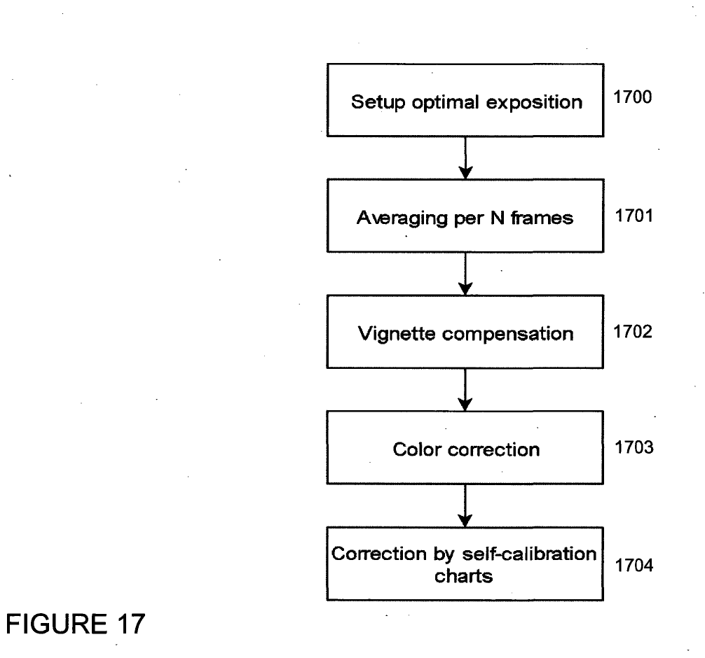

FIG. 17 is a flow diagram summarizing the distortion compensation algorithm. An optimal exposition is first set up (1700) to provide an increase in the signal to noise ratio of the input image. The input image is then averaged over N frames (1701) to help reduce any statistical measurement errors. The intensity level is aligned at all points of the image read-out using the 2D vignette compensation matrix previously stored during the calibration setup (1702) on the cartridge or cartridge carrying adaptor memory. Color correction (1703) is performed and the image intensity is then corrected using the device's reference zones (1704) by comparing the reference zones with the previously stored calibration results.

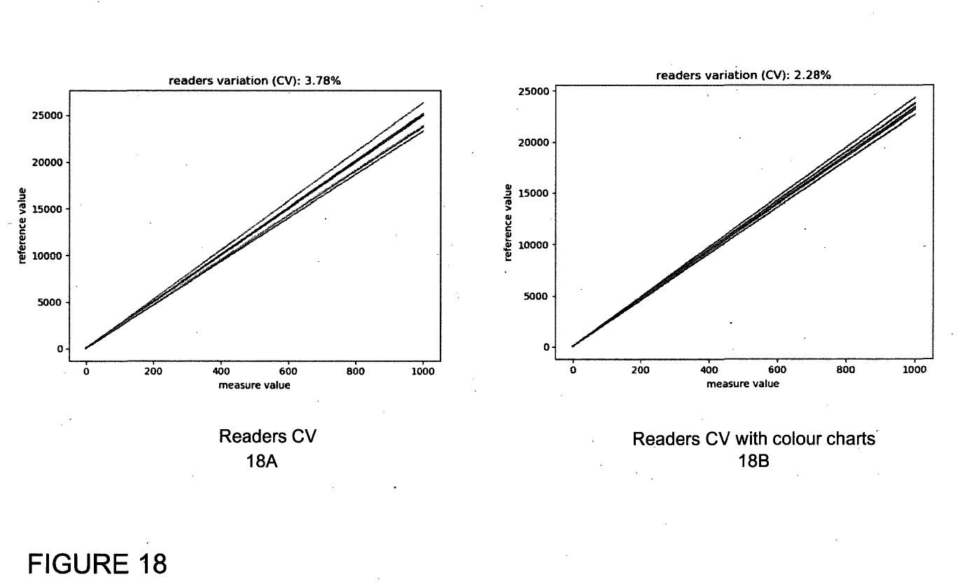

FIG. 18 shows a comparison of results obtained with 8 LF test readers without colour charts (18A) and with 8 LF test readers including colour charts (18B). Tests were performed using control cartridges with predefined results levels. A coefficient variation (CVrr) of 3.78% was obtained between the 8 readers without colour charts while a coefficient variation of 2.22% was obtained with the 8 LF test readers including colour charts. The deviation between the readers including colour charts therefore reduced by around 40%.

5. Image Correction

The coefficient variation from test results obtained from a single reader (CVr) is minimized using an image correction algorithm in order to maximize the signal to noise ratio of the captured image signal.

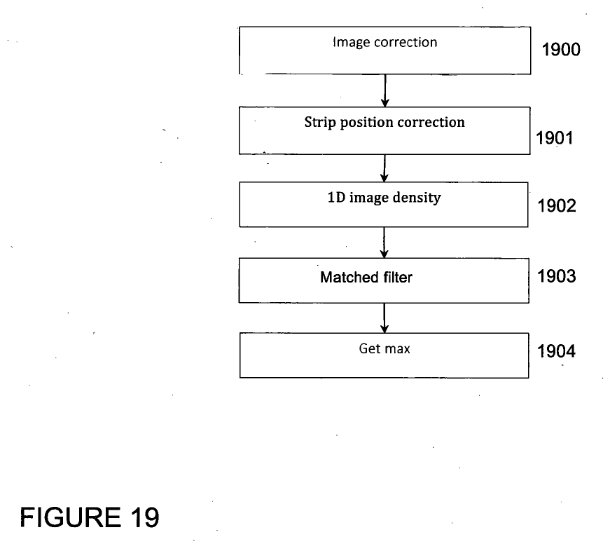

FIG. 19 shows a flow diagram illustrating key steps of the image correction algorithm. The strip position in relation to the cartridge is determined as well as the position of the lines or dots within the strip. The strip position is first corrected in order to adjust from any deviation from the standard position. The 1D image intensity profile is then determined (1902) and a matched filter is then applied for each line of the test strip (1903) and the maximum level of each line is obtained using a peak detector (1904).

The parameters of the matched filter, such as its width and shape, are determined using a statistical model derived from empirical measurements obtained from a large number of test results performed from different known concentrations.

Alternatively, a machine learning algorithms trained from empirical measurements data can also be used to obtain the parameters of the matched filter corresponding to the intensity profile of a specific line(s)/dot(s) of a test strip.

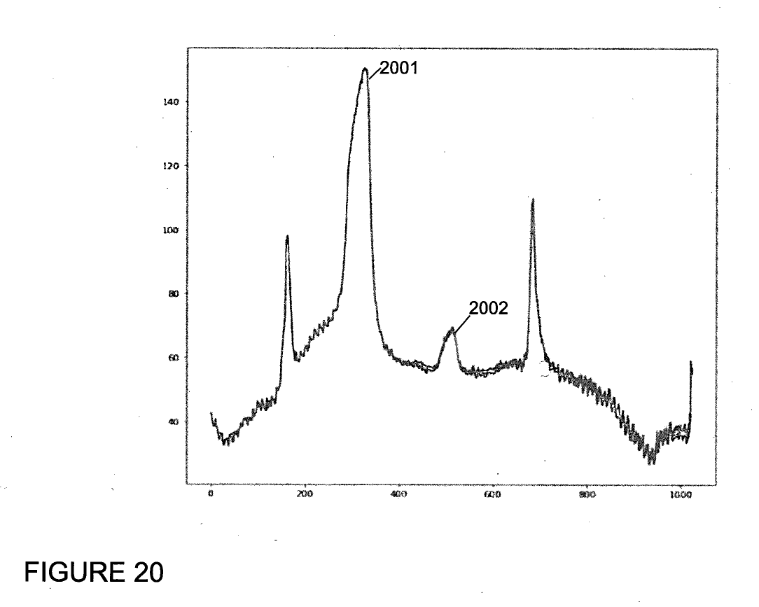

FIG. 20 shows a plot of the image intensity profile of a test strip as a function of the pixel position on the strip. The locations of the intensity profile of the test line (2001) and of the control line (2002) are shown.

From the analysis of the intensity profile of the test line (2001), the parameters of the matched filter for the test line are determined. Similarly, the analysis of the intensity profile of the control line provides parameters of a matched filter for the control line.

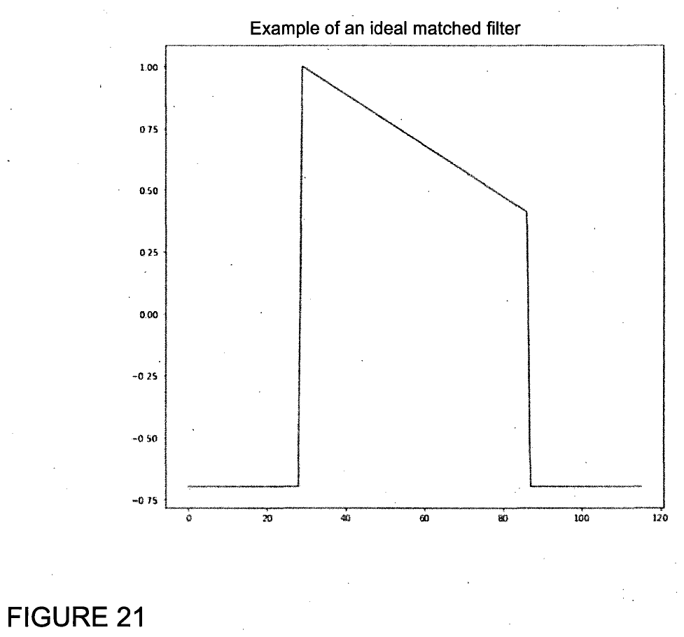

FIG. 21 shows an example of an ideal matched filter corresponding to the intensity profile of the test line determined using a statistical model.

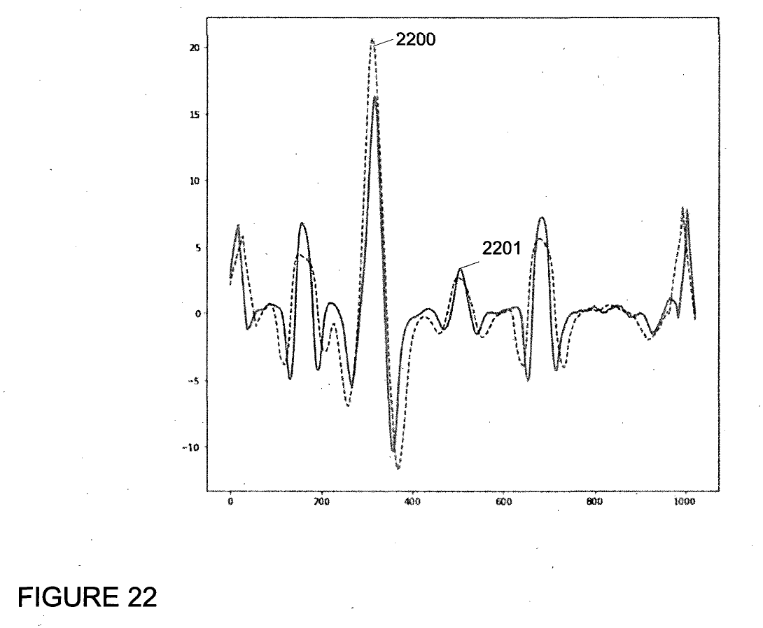

FIG. 22 shows in dashed line (2200) plot of the image intensity profile of the test strip from FIG. 20 which has been convoluted with the matched filter determined for the test line. The continuous line (2201) is a plot of the image intensity profile of the test strip convoluted with a matched filter determined for the control line. The maximum intensity values corresponding to the position of the test line and of the control lines are then extracted.

By using a matched filter an increase in test results accuracy (around 1 to 2%) is observed as compared to results obtained without using the matched filter for this specific test.

6. Imaging Sensor Module

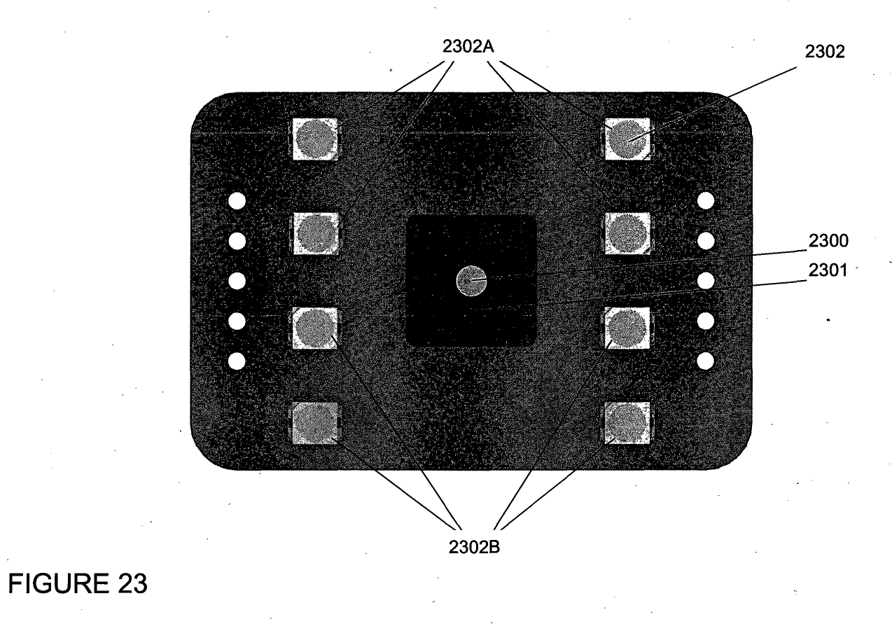

FIG. 23 shows a diagram of the image sensor module. The imaging sensor is a colour imaging sensor that includes a lens (2300), a camera module (2301) and a plurality of LEDs disposed near the lens (2302). For example, 8 LFDs may be disposed around the lens with a row of 4 LEFDs located on top of the lens (2302A) and a second row of 4 LEDs located under the lens (2302B). Each LED crystal includes 4 colours RGBW. Additionally a UV LED light may also be used.

The imaging sensor, in combination with broadband emission LEDs, allows for the reading of all existing test and or control line colors or color pads (for color-based tests), and provide for the acquisition of high density multiplexed tests in a stable light conditions.

Preferably the lens is a wide angle lens, such as a wide angle lens with an angle of view of approximately 60 degrees or more.

Cartridge or cartridge carrying adaptor may also have specific test information written or engraved on them, such as any test parameters required or a QR code. A lens with an angle view of approximately 60 degrees or more may be used to read-out such information.

The lens images a substantial part of the 2D surface of the lateral flow test strip onto the colour imaging sensor.

The image processing software then identifies patterns in the test lines and/or dots or other distributions in the test strip and compensates for distortions, non-uniformities or anomalies in the lines and/or dots or other distributions in the test strip.

The lens is a commodity non-custom lens and the sensor is a commodity CMOS sensor that inherently introduces distortions when creating an image of the substantial part of the 2D surface.

Preventing lens reflection: lens reflection may potentially degrade the quality of the image. In addition wet samples may suffer from image glare. Hence one or more LEDs may be switched on or off in order to reduce the effect of lens reflection and image glare. As an example, the top four LEDs (2302A) may be switched on in order to read the upper half of the sample test as shown in FIG. 23, with the corresponding bottom four LEDs switched off. The bottom four LEDs (2302B) may then be switched on in order to read the bottom half of the sample test with the top four LEDs switched off. By selectively turning on or off different LEDs, the sample test may be measured more accurately. Bleaching out of the image, or unwanted reflections, is avoided.

The number of LEDs as well as the form the LED matrix may vary. However the position of the LEDs is chosen in order to improve the sensitivity of test results.

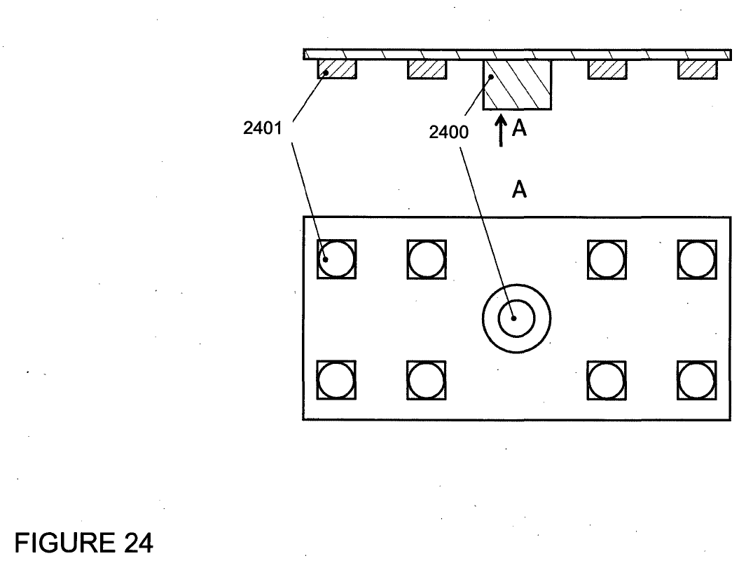

FIG. 24 shows a cross section view and a top view with another configuration for an image sensor module comprising a camera module (2400) and a plurality of LEDs disposed near the camera module (2401).

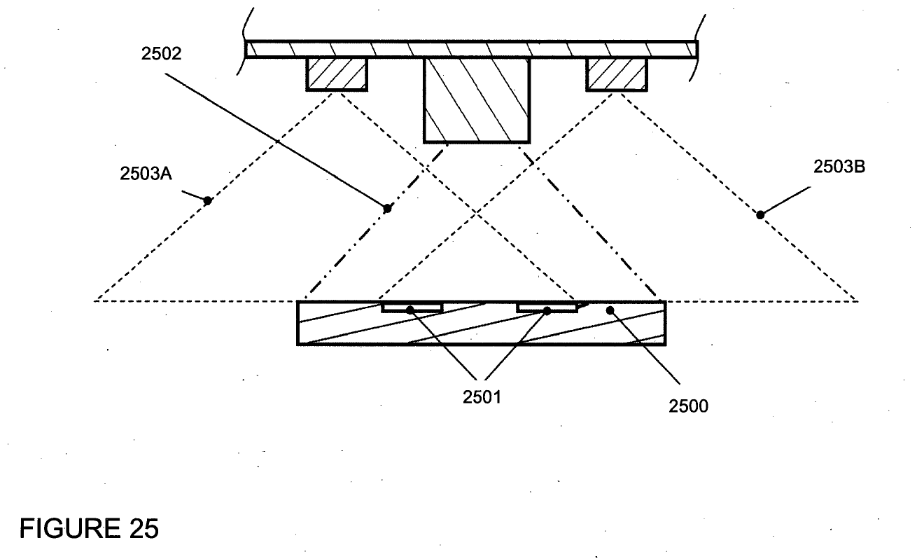

FIG. 25 shows the camera module imaging a cartridge (2500) comprising test strips (2501). The field of view of the camera (2502) as well as the area illuminated by the LEDs (2503A-2503B) are shown.

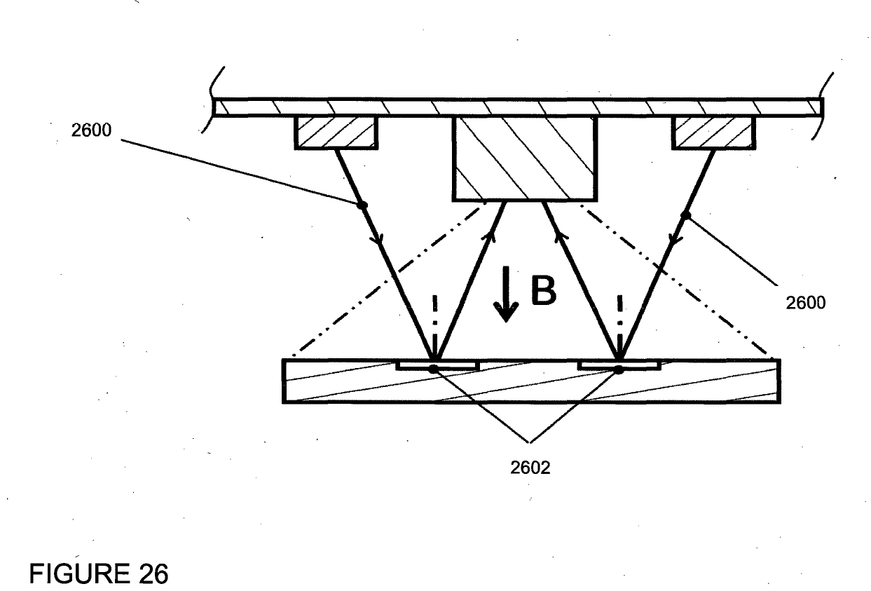

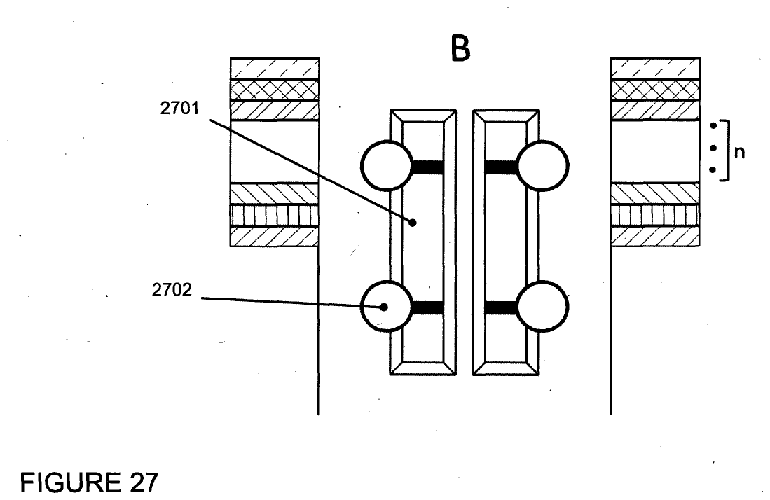

As shown in FIG. 26, when all the LEDs are switched on (2601), image glare may appear (2602). FIG. 27 shows a top view of a test strip (2701) in which image glares from the LEDs are present (2702).

Glare occurrence greatly depends on the LEDs positioning—close to the camera's centre the test results read out may suffer greatly from strong reflections (glares). Far from the camera centre it is possible to reduce glares but the shadows from plastic will be stronger, and will result in reduced accuracy of the test result.

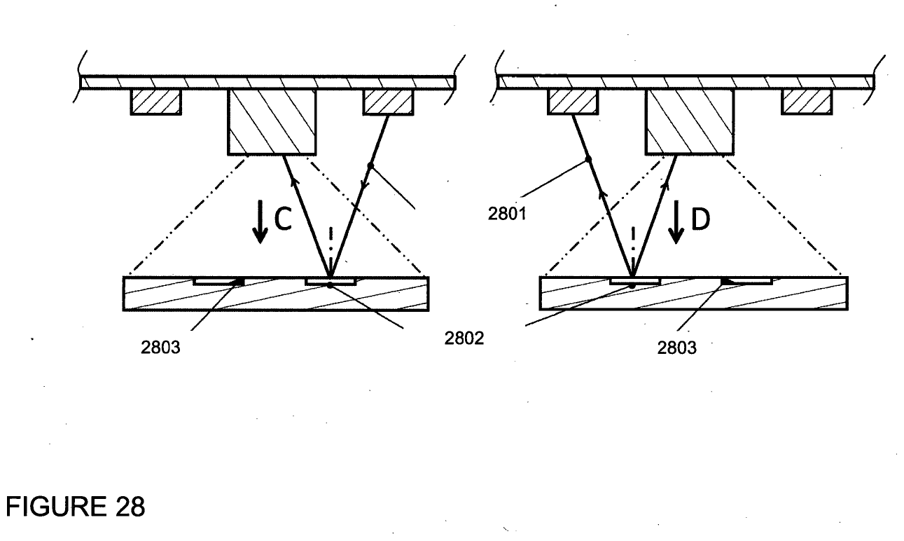

As illustrated in FIG. 28, when a LED is switched on (2801) to illuminate a cartridge with two test strips, glare may appear on one of the test strip (2802) and shadow may appear on the other test strip (2803).

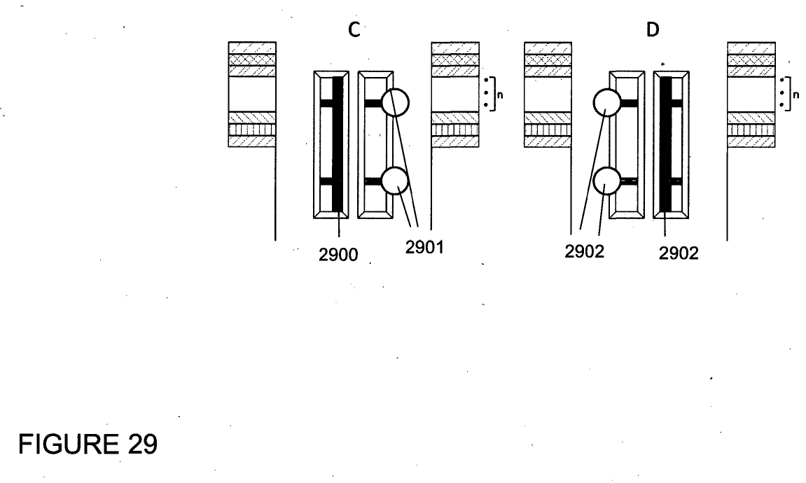

The images resulting from the illuminations of C and D from FIG. 28 in which the LEDs are switched on and off sequentially are shown in FIG. 29. The resulting images include unwanted glares and shadows appearing on the cartridge with two test strips from the illuminations C and D of FIG. 28 is shown in FIG. 29.

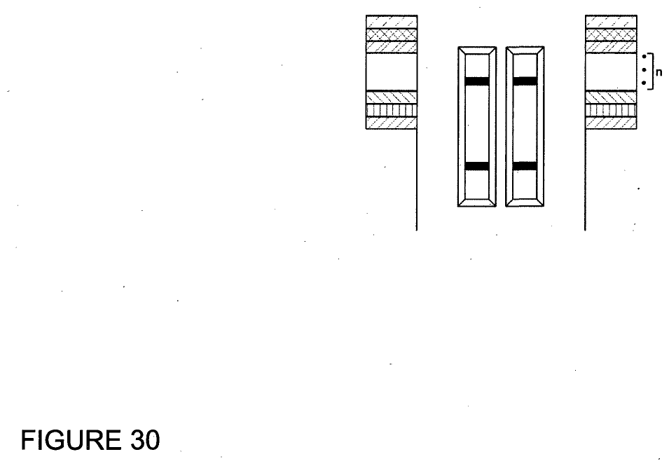

The left and right images are combined and averaged in order to result in the image shown in FIG. 30. Reference zones that are present inside of the reader may also help to compensate color differences.

7. Kinetic Data

Capturing and analysing the rate of change in the test strip over time (i.e. data from a kinetic analysis or ‘kinetic data’) may improve the accuracy of the results as compared to analysing a captured image of the test strip at a single point in time such as when the exposition time is finished. The final image of the test lines and/or dots may therefore be captured alongside the progress of the test over time.

Hence the cartridge may also store kinetic data in memory. This can be for example in the form of a statistical model. This can be provided to a remote server to enable a statistical model or library of kinetic data, or associated test performance data, to be constructed.

The imaging sensor module captures images of one or more test strips, at time intervals over a certain time period. The time intervals and time period may be preprogrammed on the diagnostic device itself, or may be included in the kinetic data stored in the cartridge memory.

Analysing the test strip over time also allows errors to be identified early. For example, the kinetic data may reveal that an error has occurred, e.g. the buffer has not been used with the sample. In this case, the user would be notified that the results should be discarded.

Analysing the progress of the test strip over time, also allows a reduction in the time needed to provide test results. By storing a kinetic statistical model, it is possible to forecast what the final curve will look like, after a much shorter time period.

Further, analysing the progress of the test strip over time, also provides useful data on the test performance, for example by identifying the start time and end time of the test:

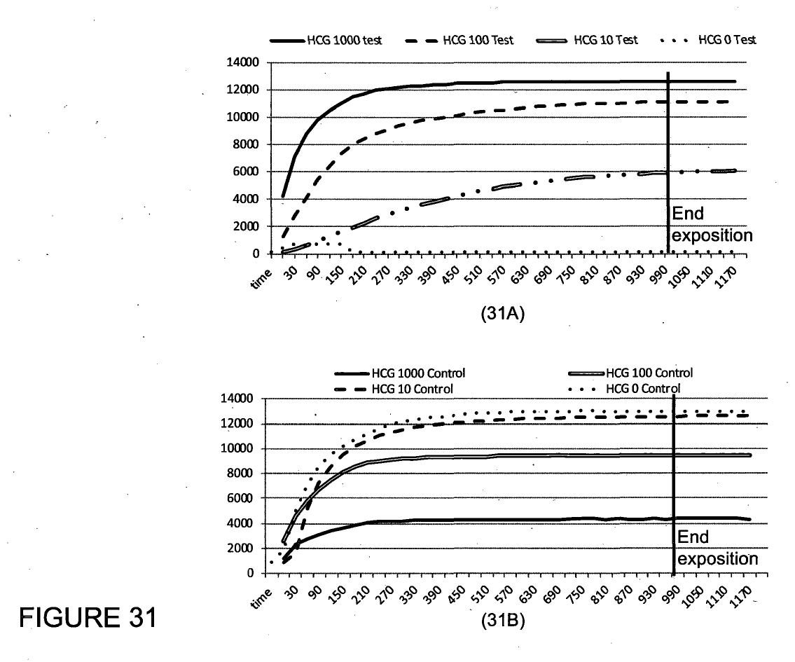

FIG. 31 shows a plot with kinetic results performed on a pregnancy test strip (hCG). An hCG test strip with four different concentration levels are analysed over time. The results show the analysis of the progress of the test line (31A) and of the control line (31B) as a function of time.

From analysing the progress or rate of change of the lateral test strip as a function of time, the following may be determined:
Velocity of the sample;
Amount of buffer used;
Proportion of buffer vs. sample used;
Time to provide a test result depending on the type of membrane used;
Time to provide a test result depending on the test ran.

By storing and analysing these parameters, test results can be obtained with higher accuracy. The analysis of large amount of experimental data obtained by LF-test readers are used to provide an intelligent feedback and to improve the efficiency of the LF-test reader.

The intelligent feedback can be used to include firmware updates or to recommend specific test parameters allowing test manufacturers to design improved test procedures such as for optimizing a test performance duration or to reduce results variation. For example, feedback may be given to test manufacturers on the quality of their test strips.

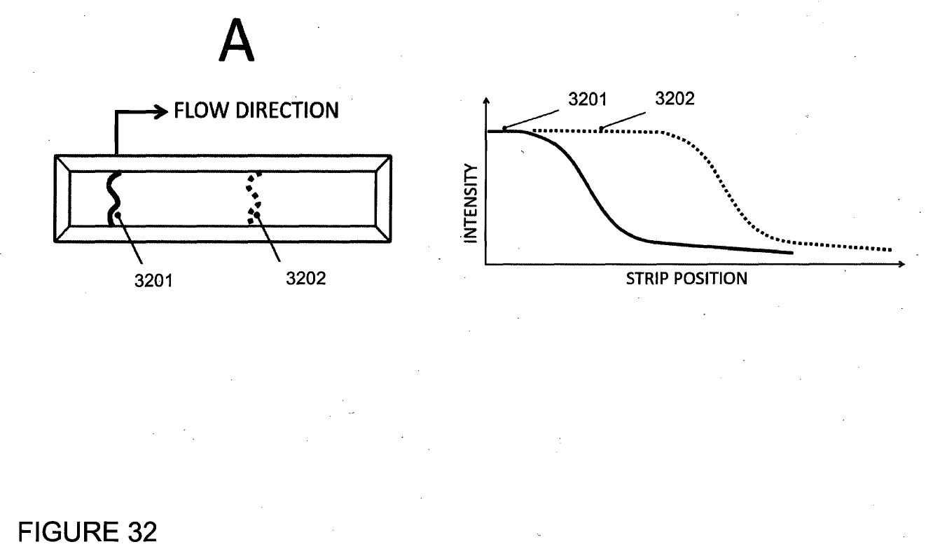

FIG. 32 shows the example of sample flow on the membrane at two different time intervals. 3201 shows the flow at a first point in time t1 and 3202 at a second point in time t1+Δt. By that means the flow rate can be calculated.

At the first point in time t1 (3201), the flow rate is a function of the sample viscosity and is analysed to extract the proportion of buffer vs. sample used. This is possible due to the analysis of a large number of empirical measurements of the specific test results performed in a laboratory environment.

8. Active Adapter for Electrochemistry and Reflectance Photometry

As discussed above, the LF test reader operates with any rapid lateral flow assays, test strips, dipsticks test or any other tests that are commercially available.

To date, there are no other devices on the market combining a readout of rapid tests using different measurement technologies. The cartridge carrying adaptor can be configured to hold electrochemical and reflectance photometry based tests.

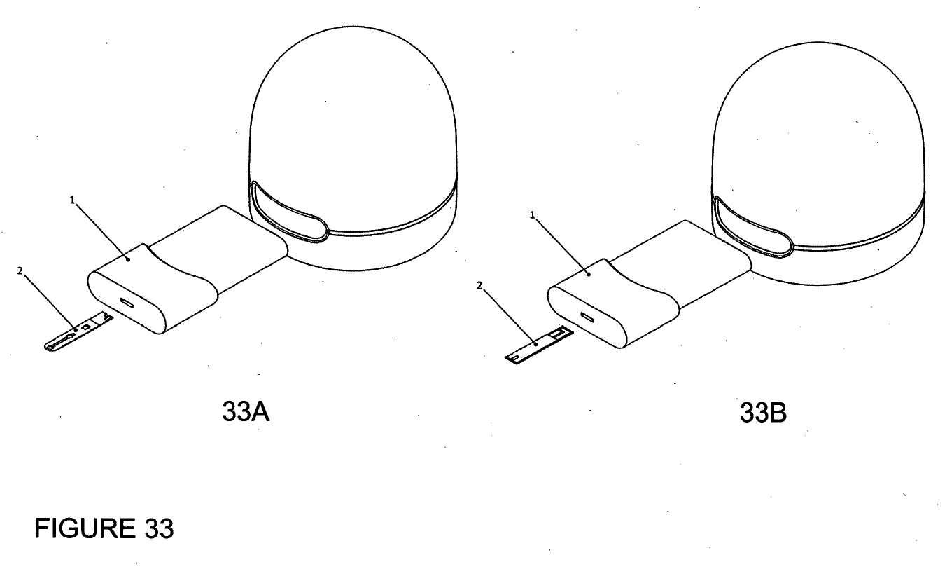

As an example FIG. 33A shows the LF test reader with a cartridge carrying adaptor configured to hold photometric test in order to perform reflectance photometry tests. FIG. 33B shows the LF test reader with a cartridge carrying adaptor configured to hold an electrochemical test in or to perform tests based on electrochemistry.

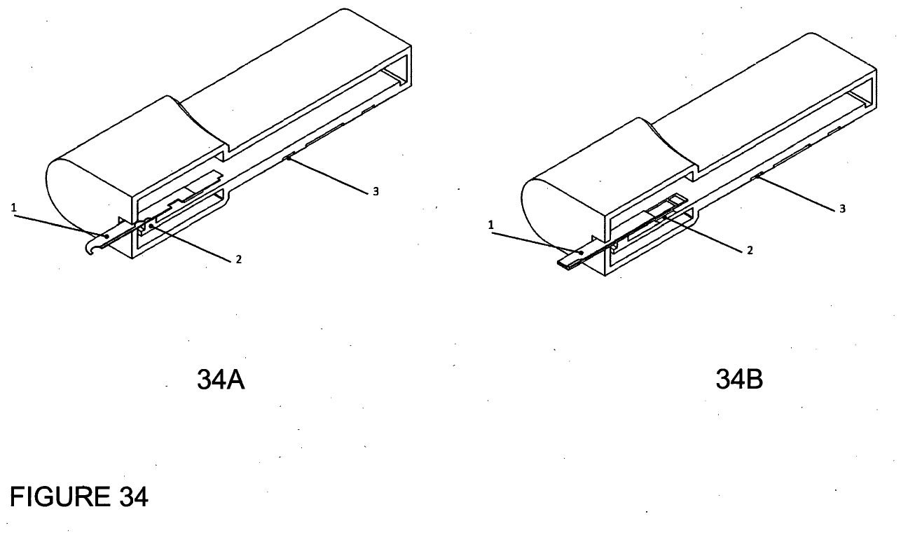

With reference to FIG. 34A, the cross section of a cartridge carrying adaptor for photometric tests is shown with an inserted photometric test (1). The read-out of test result is performed by photodiode (2) which is charged from the reader via NFC (3). The obtained results are then send to the reader via the NFC interface.

With reference to FIG. 34B, the cross section of a cartridge carrying adaptor for electrochemical tests is shown with an inserted electrochemical test (1) is shown. The read out of the test result is performed by electrodes and transmitted to the reader via NFC interface (2).

Using low cost active adaptors with various sets of internal actuators and sensors makes it possible to use just one device performing different types of tests and to store all tests results in a safe cloud.

Temperature control adaptor: some tests, such as photometric test have to be performed in a controlled temperature mode. The active temperature control adaptor includes a thermo sensor or any other heating elements, such as a Peltier element, to carry out the control of the temperature. The energy required to read out the tests can be transmitted to the active adaptor through an RFID transmitter. Read-out is performed using one or a combination of the following, included in the adaptor: a photo-diode, one or more LEDs (of UV, visible or infrared wavelengths) or a camera.

Adaptor for dipstick tests: dipstick tests, such as urine tests, include several squares of different colors attached to it. When the number of squares is large, such as greater than 4, the active adaptor may include a mechanical fixture arranged to move the cartridge along the adaptor when the cartridge is inserted inside the reader. Hence portions of the dipstick test are moved along the adaptor and read out one at a time by the reader. A portion may include for example four squares of the dipstick test.

Hence, the results from photometric tests, electrochemical tests as well as from lateral flow immunoassay test strips, micro-array immunoassays or any other tests may all be stored by the reader for combined analysis. The combined results may then also be stored to a cloud platform collecting test data and patients' or users' personal profiles.

9. Application Running on a Connected Device.

The portable diagnostic device exchanges data with an application running on a connected device. The connected device then connects over a secure link via the Internet or a cellular network to a remote, cloud based server. The connected device may be for example: a desktop, laptop, tablet, smartphone, smart watch, or any other wireless electronic device.

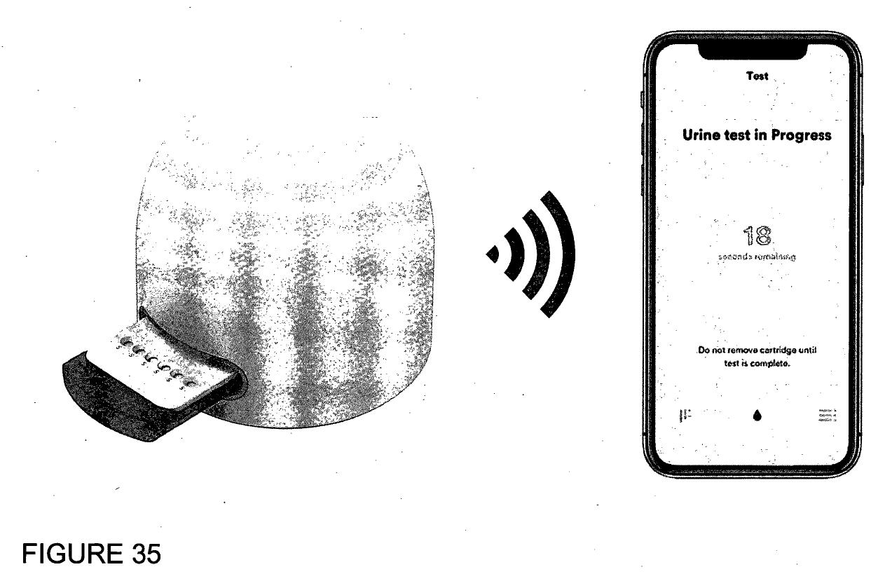

In FIG. 35, the application running on the connected device displays a progress page while the diagnostic device is reading out the cartridge test results. The diagnostic device has identified the test strip that was slid into it and the user is notified of the remaining time before results are displayed.

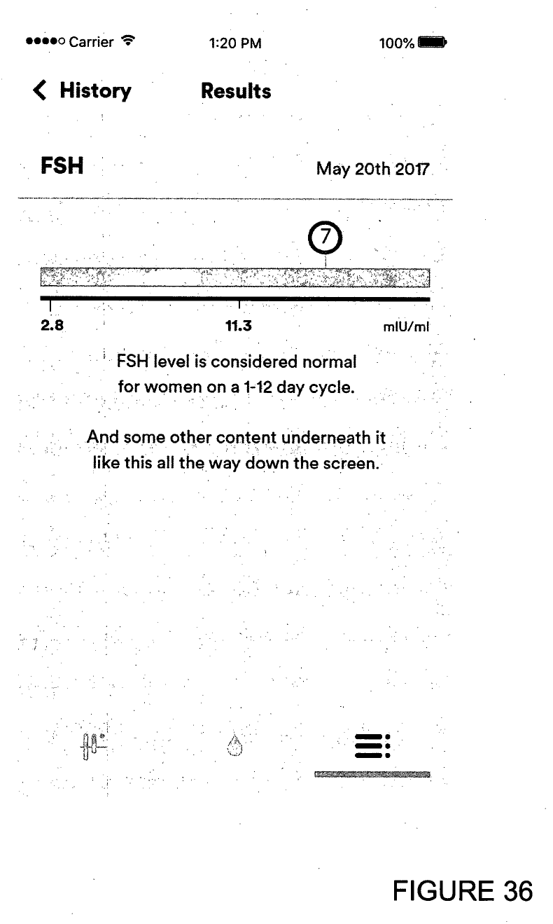

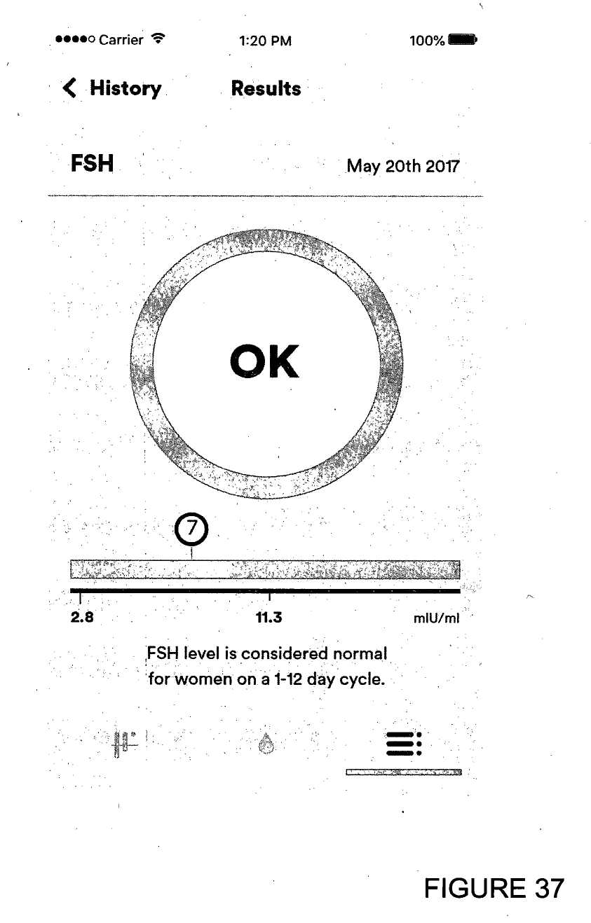

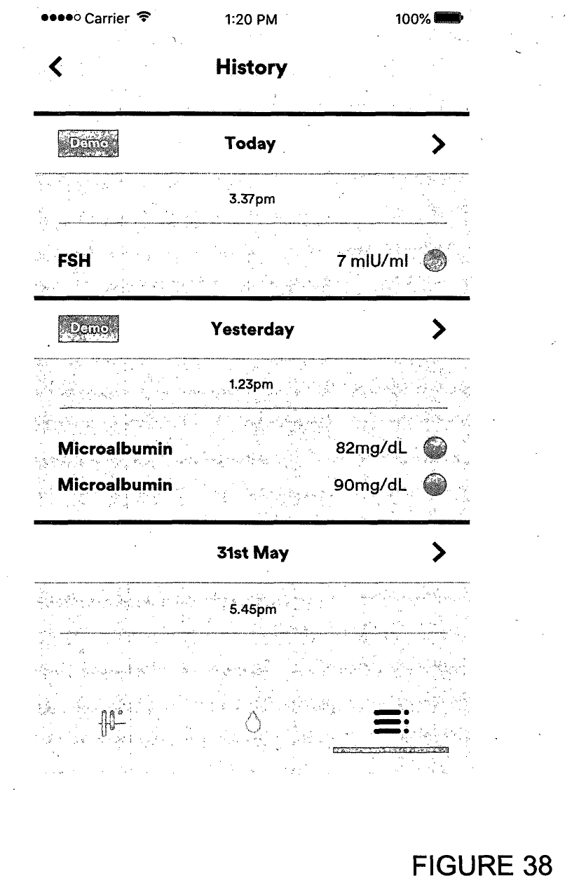

FIG. 36 shows a page displaying an example of FSH test results. FIG. 37 shows a page displaying an example of FSH test results. FIG. 38 shows a history page with a summary of the day's test results.

The application allows for visualizing the test results and the test results history such that they are easy to interpret. In addition the application may also store patient demographics, test diagnostics and medical data.

10. Cloud Platform

The image processing software uses a cloud-based model, statistical model, library or statistical library of patterns. The model also stores ways to compensate for distortions, non-uniformities or anomalies in the lines, dots or distributions in the test strip.

Machine learning techniques are used to build and improve the model or library of patterns. In turns, the model is used to develop processing algorithms and to update the software for improving accuracy.

The cloud-based model or library is stored on a remote, secure cloud platform. The diagnostic device and the connected devices all have access to the cloud platform and the cloud-based model or library.

The application sends data to the cloud platform which stores test data and patients' or users' personal profiles.

The secured cloud platform may be hosted in a cloud data-center, like Amazon Web Services (RTM), Microsoft Azure (RTM) or any other PaaS provider compliant with HIPAA and/or other regional health data security regulations.

The cloud platform further enables:
Encrypted wireless connectivity with the application and the diagnostic device for patient tests data, demographic and medical diagnostic data management;
The ability to build real-time health maps for certain geographical areas;
Provide data analysis capabilities for laboratory R&D processes and test development;
Manufacturing and supply chain management services, including inventory of devices and batches of test cassettes;
Remote monitoring of the diagnostic device health status, enabling proactive support of customers;
Flexible and secured EHR systems integration capabilities;
White-labeling option.

11. Test Development Kit for Tests Developers and Manufacturers

A desktop application is made available for tests developers and manufacturer in order to process and analyze test data during tests development and validation.

The test development kit available for example on a desktop application includes, but is not limited to, the following features:
Processing and analysis of test data during test development and validation;
Setting calibration curves, marker location windows, preparation time for batches of tests during the development or manufacturing process;
Encrypted wireless connectivity with the Cloud Platform, for tests or batches of test data uploads.

12. Data Privacy Engine

In the modern world the accessibility of the mobile devices data storage is very significant provided all the data is safely stored in a de-identified and an anonymised way.

Commercially available readers are not always able to store the results in the common repository, requiring the user either to write down the results or to print them out. Data collection is very significant for the modern medicine, that is why our device is not equipped with display, motivating the customer to use the application running on a connected device, carrying out the liaison between the User and measurement results, to enforce uploading the data to the cloud for the subsequent data analysis

The system stores de-identified, anonymised data. The de-identified, anonymised interpretation of tests is stored as metadata on the cartridge adapter with NFC tag or QR code. The stored metadata is therefore always available to the user without the need to be connected to internet.

The test identification including the device ID and the time the test was performed is stored.

Data including gender, age, weight, race or any other personal data can be added after a user's registration in the application for further analysis.

13. Simplified User Interaction with the LF Test Reader

Confirm pairing by tapping the device: in a laboratory environment, a large number of portable diagnostic devices may be available. From the connected device application, it is possible to select which diagnostic device to pair to the connected device and to run the next diagnostic from.

Once selected on the connected application, the diagnostic device indicates that pairing can be initiated, for example the LED ring on the outside of the diagnostic device starts blinking in blue. The user can then confirm pairing and initiate the pairing of the diagnostic device with the connected device by tapping the device. By adding this simple confirmation step, incorrect device pairing can be reduced.

A diagnostic result can be run automatically following only a one-step user interaction. The device is configured to automatically run a diagnostic result following a one-step user interaction with the device, such as:
The diagnostic test result is automatically performed once a test cartridge or cartridge carrying adaptor with the test cartridge is slid and inserted in place inside the portable diagnostic device. This is the case when the required exposition time of the test has passed. This is automatically performed without the user having to physically turn on the diagnostic device.
When a cartridge is already inserted in the portable diagnostic device, the diagnostic test result is automatically performed after tapping the portable diagnostic device. In this case the reader awaits for a required exposition time and then perform the read-out the test result.

In both cases mentioned above the device is connected to a application.

The diagnostic device automatically turns on, self-calibrates and reads the cartridge test results. The results are automatically stored and/or displayed on a connected application.

In comparison with other devices, the operation of the device has been greatly simplified with the number of steps required from a user to run a test reduced. This in turns produce faster results and avoid additional settings.

Appendix 1: Key Features

This section summarises the most important high-level features (A->N); an implementation of the invention may include one or more of these high-level features, or any combination of any of these. Note that each high-level feature is therefore potentially a stand-alone invention and may be combined with any one or more other high-level feature or features or any of the ‘optional’ features; the actual invention defined in this particular specification is however defined by the appended claims.

A. Software Compensation for Lens Distortions

A portable, rechargeable lateral flow test strip immunoassay in vitro diagnostic device, including a colour imaging sensor for imaging a lateral flow test strip, including a lens that images a substantial part of the 2D surface of the lateral flow test strip onto the colour imaging sensor and image processing software that identifies patterns in the test lines and/or dots or other distributions in the test strip and compensates for distortions, non-uniformities or anomalies in the lines and/or dots or other distributions in the test strip.

Optional features in an implementation of the invention include any one or more of the following:
Lens is a commodity non-custom lens and commodity CMOS sensor that inherently introduce distortions when creating an image of the substantial part of the 2D surface.
BOM cost of the lens and CMOS sensor is $7 or less, such as $5 or less.
CMOS sensor has a resolution of 2 megapixels or less.
Lens is a wide angle lens, such as a wide angle lens with an angle of view of approximately 60 degrees or more
The device allows for the reading and testing of lateral flow test strip with several lines and/or dots distributed across the 2D surface.
Image processing software uses a cloud-based model, statistical model, library or statistical library of patterns, and also ways to compensates for distortions, non-uniformities or anomalies in the lines, dots or distributions in the test strip.
Image processing software uses machine learning techniques to build its model or library of patterns and its ways to compensates for distortions, non-uniformities or anomalies in the lines, dots or distributions in the test strip.
Image processing software correlates distortions, non-uniformities or anomalies in the lines or distributions in the test strip to variables, such as batch numbers, and environmental factors.
Digital filters are applied to the captured image signal of the test strip.
Connected devices all have access to the cloud-based model or library
N-LEDs of different colours (Red, Green, Blue and White or UV LEDs) are used to illuminate the test strip. These can be n-LEDs above the strip, or below.
Imaging sensor images substantially the entire surface of the test strip; or at least 90% of the entire surface of the test strip; or at least 80% of the entire surface of the test strip; at least 70% of the entire surface of the test strip, or at least 60% of the entire surface of the test strip.
A distortion compensation algorithm taking into account the lens parameters, the location of the LEDs in relation to the lens and the position of the test strip in relation to a cartridge.
The distortion compensation algorithm further takes into account the results of a calibration obtained by imaging a colour and/or white charts.

B. Imaging Adjacent Test Strips

A portable, rechargeable lateral flow test strip immunoassay in vitro diagnostic device, including a colour imaging sensor for imaging the lateral flow test strip, including a lens that simultaneously or near simultaneously images a substantial part of the 2D surface of two or more adjacent immunoassay test strips.

Optional features in an implementation of the invention include any one or more of the following:
Lens is a commodity non-custom lens and commodity CMOS sensor that inherently introduce distortions when creating an image of the substantial part of the 2D surface.
BOM cost of the lens and CMOS sensor is $7 or less, such as $5 or less.
CMOS sensor has a resolution of 2 megapixels or less.
Lens is a wide angle lens, such as a wide angle lens with an angle of view of approximately 60 degrees or more

C. Using a Wide Angle Lens

A portable, rechargeable lateral flow test strip immunoassay in vitro diagnostic device, including a colour imaging sensor for imaging the test strip, including a wide angle lens that images a substantial part of the 2D surface of the immunoassay test strip onto the colour imaging sensor.

Optional features in an implementation of the invention include any one or more of the following:
Wide angle lens has an angle of view of at least 60 degrees.
Lens is a commodity non-custom lens and commodity CMOS sensor that inherently introduce distortions when creating an image of the substantial part of the 2D surface.
BOM cost of the lens and CMOS sensor is $7 or less, such as $5 or less.
CMOS sensor has a resolution of 2 megapixels or less.
Device images a uniformly coloured test region, e.g. a white region, to measure and compensate for lens vignetting.

D. Using Several Camera Modules

A portable, rechargeable lateral flow test strip immunoassay in vitro diagnostic device, including two or more camera modules, each camera module including an imaging sensor and a lens that images a part of the 2D surface of the immunoassay test strip onto the imaging sensor, and in which the two or more camera modules have different specifications.

Optional features in an implementation of the invention include any one or more of the following:
a lens is a commodity non-custom lens and commodity CMOS sensor that inherently introduce distortions when creating an image of the substantial part of the 2D surface.
The camera modules have different specifications, such as a different FOVs, resolutions, pixel sizes, sensitivities or filters.
A first camera module includes a colour imaging sensor and a second camera module includes a black and white imaging sensor.
Each camera module is configured to image a specific portion of the test strip or to image a specific portion of a cartridge carrying the test strip.
Each camera module is configured to image a specific type of test trip.
One camera module includes a global shutter to read out fluorescent-based lateral flow test.

E. Automatic Self-Calibration Using Colour and/or White Charts Built Into Part of the Device and on a Dedicated Cartridge or Adaptor

A portable, rechargeable lateral flow test strip immunoassay in vitro diagnostic device, including a colour imaging sensor for imaging the test strip, including a wide angle lens that images a substantial part of the 2D surface of the test strip onto the colour imaging sensor and that is configured, to automatically self-calibrate in the colour space by imaging a colour and/or white chart, in which the colour and/or white charts are built into part of the device and are also present on a dedicated cartridge or cartridge carrying adaptor that is slid into the device.

Optional features in an implementation of the invention include any one or more of the following.
Imaging software uses the data from the calibration process to compensate for temperature dependent drift in the colour mapping of the imaging sensor.
Imaging software uses the data from the calibration process to ensure that data from multiple different devices analyse consistently.
The calibration curve associated with imaging a specific test strip held in a cartridge is written to a memory or record on or associated with that cartridge, including a QR code engraved or printed on the cartridge or an RFID tag or other memory on the cartridge or adaptor.
Imaging the white chart from a dedicated calibration cartridge slid into the device determines the following calibration parameters: exposition value and/or vignette compensation parameters.
Imaging the colour charts from a dedicated calibration cartridge slid into the device determines the following calibration parameters image scale factor, a color correction matrix and/or the image sensor's displacement.
Calibration parameters are transmitted and stored into a cloud for subsequent analysis of test strips.

F. Analysing Lateral Flow Strips and Also Micro-Arrays

A portable, rechargeable lateral flow immunoassay test strip in vitro diagnostic device, including a colour imaging sensor for imaging the test strip, including a lens that images a substantial part of the 2D surface of the immunoassay test strip onto the colour imaging sensor and that is configured to receive and also analyse cartridges for both lateral flow immunoassay test strips and also micro-array immunoassays.

G. Illuminating with a UV Light Source

A portable, rechargeable lateral flow immunoassay test strip in vitro diagnostic device, including a colour or a black and white imaging sensor for imaging the test strip, including a wide angle lens that images a substantial part of the 2D surface of the immunoassay test strip onto the colour imaging sensor and that includes a UV light source to illuminate the immunoassay test strip.

Optional feature in an implementation of the invention includes any one or more of the following:
A UV filter is included in front of the wide angle lens.

H. Cloud-Connectivity via Bluetooth to a Smartphone

A portable, rechargeable lateral flow immunoassay test strip in vitro diagnostic device, including a short range, secure communications link or interface that enables the device to exchange data with an application running on a smartphone or other wireless device, and the smartphone or other wireless connected device then connects over a secure link via the internet or a cellular network to a remote, cloud-based server.

Optional features in an implementation of the invention include any one or more of the following:
The diagnostic device accepts different types of cartridge carrying adaptor that are configured to hold any commercially available test, such as electrochemical test, reflectance photometry based test, rapid lateral flow assay, test strip or dipsticks.
a cartridge carrying adaptor includes various sets of actuators or sensors in order to perform the different tests.
a cartridge carrying adaptor includes a temperature control system for controlling the temperature in the cartridge when a test is being read out.
a cartridge carrying adaptor includes a mechanical fixture arranged to move a dipstick test along the cartridge carrying adaptor, such that portions (i.e four squares at a time) of the dipstick test are read out sequentially by the device.
the results of the different types of test are transmitted, analysed and combined on the server.

I. Cartridge Stores the Kinetic Data and the Calibration Curve

A cartridge for a portable, rechargeable lateral flow immunoassay test strip in vitro diagnostic device, the cartridge including or associated with a read/write memory to which is written, in normal operation by the device, the kinetic data and the calibration curve obtained when the device images a test strip mounted on or associated with that cartridge.

Optional features in an implementation of the invention include any one or more of the following.
Calibration curve is a colour calibration curve.
Cartridge is configured by shape and size to slide into an aperture in the device.
Read/write memory is an integral part of or attached to the cartridge.
Read/write memory is an integral part of or attached to an adaptor for the cartridge.
The cartridge memory has written to it one or more of the following: the test type, expiration date, test date, test time, test calibration date, calibration curve for this batch or lot number, test manufacturer, test manufacturer address, test manufacturer contact information.
The calibration curve is updated when a subsequent test strip result is obtained.
The calibration curve is updated from the results stored on the diagnostic device or on a cloud-based library that is accessible by the diagnostic device.

J. Cartridge Accepts a Lateral Flow Test Strip and Also a Micro-Array Test Strip

A cartridge for a portable, rechargeable lateral flow immunoassay test strip in vitro diagnostic device, the cartridge configured to receive either a lateral flow test strip or a micro-array test strip.

K. Cartridge is Locked with a Crypto-Chip to Ensure Authenticity

A cartridge for a portable, rechargeable lateral flow immunoassay test strip in vitro diagnostic device, the cartridge including a secure memory chip or device and a communications chip or device, the secure memory storing a unique identification code or crypto-key or number and the cartridge configured to undertake a handshake or other protocol in which the unique identification code or crypto-key or number is checked and verified as authentic or otherwise genuine.

L. LEDs are Switched On or Off Selectively in Order to Improve Accuracy of the Results

A portable, rechargeable lateral flow immunoassay test strip in vitro diagnostic device, including a colour imaging sensor for imaging the test strip, including a lens that images a substantial part of the 2D surface of the immunoassay test strip onto the colour imaging sensor and that includes multiple LEDs of different colours used to read the test strip, in which the device can be programmed to select only a set of LEDs to be turned on, in order to image a specific region of interest of the 2D surface of the immunoassay test strip onto the colour imaging sensor.

Optional features:
in which a set of LEDs located above the lens is turned off, and a set of LEDs located below the lens is turned on, in order to image the bottom region of the test strip.
in which a set of LEDs located below the lens is turned off, and a set of LEDs located above the lens is turned on, in order to image the top region of the test strip.
in which the results obtained from imaging the bottom region and top region of the test strip are combined.

M. Kinetic Analysis of the Test Strip to Provide Faster Results

A portable, rechargeable lateral flow test strip immunoassay in vitro diagnostic device, including a colour imaging sensor for imaging a lateral flow test strip, including a lens that images a substantial part of the 2D surface of the lateral flow test strip onto the colour imaging sensor, and in which the device is configured to capture the progress or change or rate of change of the lateral flow test strip as a function of time (‘kinetic data’).

Optional features:
Includes an image processing software that identifies as a function of time patterns in the test lines and/or dots Or other distributions in the test strip and compensates for distortions, non-uniformities or anomalies in the lines and/or dots or other distributions in the test strip.
kinetic data associated with imaging a specific test strip held in a cartridge is written to a memory or record on or associated with that cartridge, including a QR code engraved or printed on the cartridge or an RFID tag or other memory on the cartridge or cartridge adaptor.
Imaging software uses the kinetic data to obtain or measure the lateral flow test result.
By comparing the stored kinetic data, the device is able to forecast what the rate of change of the test strip should look like.
By analysing the progress or rate of change of the test strip as a function of time, the device is able to detect errors early.
A detected error is that the buffer has not been included with the sample.
Images of the substantial part of the 2D surface of the lateral flow test strip are captured at predefined intervals, such as every 10 or 20 seconds.
Images of the substantial part of the 2D surface of the lateral flow test strip are captured at predefined intervals and for a predefined duration, each being determined by the specific test being undertaken.
Predefined intervals and predefined duration are included in the kinetic data stored on a cartridge.
The device provides the progress or change, or rate of change of the lateral flow test strip as a function of time (‘kinetic data’) to a data analysis system that uses that kinetic data to improve the coefficients of variation of test results.
The device monitors whether or not the lateral flow test strip changes during a predefined initial period, such as the first 10 or 20 seconds, in a way that is consistent with the test operating correctly; and if no such changes are detected, then the device generates an alert.
The kinetic data is compared to a statistical library or model of kinetic data for both successful and failed tests to determine the likelihood of the test being successful, and, if the possible or likely causes of any unsuccessful test.

N. Simplified User Interaction with the Device: a Diagnostic Result Can be Run Automatically Following Only a One-Step User Interaction

A portable, rechargeable lateral flow test strip immunoassay in vitro diagnostic device, including a colour imaging sensor for imaging the lateral flow test strip, including a lens that images a substantial part of the 2D surface of the immunoassay test strip onto the colour imaging sensor and that is configured to automatically run a diagnostic result following a one-step user interaction with the device.
The diagnostic test result is automatically performed once a dedicated cartridge or cartridge carrying adaptor is slid and inserted in place inside the portable diagnostic device.
When a cartridge is already inserted in the portable diagnostic device, the diagnostic test result is automatically performed after tapping the portable diagnostic device.
When a cartridge is already inserted in the portable diagnostic device, the diagnostic test result is automatically performed when the diagnostic device is selected on an application running on a connected device.

These steps may be automatically performed without the user having to physically turn on the diagnostic device.

The diagnostic device automatically turns on, self-calibrates and reads the cartridge test results. The results are automatically stored and/or displayed on a connected application.

Note

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred example(s) of the invention, it will be apparent to those of ordinary skill in the art that numerous modifications can be made without departing from the principles and concepts of the invention as set forth herein.

1. A portable, rechargeable lateral flow test strip immunoassay in vitro diagnostic device, including a color imaging sensor for imaging a lateral flow test strip, including a lens that images a substantial part of the 2D surface of the lateral flow test strip onto the color imaging sensor and image processing software that is adapted to (i) identify patterns in the test lines and/or dots or other distributions in the test strip and (ii) compensate for distortions, non-uniformities or anomalies in the lines and/or dots or other distributions in the test strip.

2. The device of claim 1, in which the lens is a non-custom lens that inherently introduces distortions when creating an image of the substantial part of the 2D surface and the image processing software is adapted to compensate for those distortions.

3. The device of claim 1, in which the BOM (bill of material) cost of the lens and the color imaging sensor is $7 or less.

4. The device of claim 1, in which the color imaging sensor has a resolution of about 2 megapixels or less.

5. The device of claim 1, in which the lens is a wide angle lens, such as a wide angle lens with an angle of view of approximately 60 degrees or more.

6. The device of claim 1, in which the device allows for the reading and testing of lateral flow test strip with several lines and/or dots distributed across the 2D surface.

7. The device of claim 1, in which the image processing software uses a cloud-based model, statistical model, library or statistical library of patterns, and also ways to compensates for distortions, non-uniformities or anomalies in the lines, dots or distributions in the test strip.

8. The device of claim 1, in which the image processing software uses machine learning techniques to build its model or library of patterns and its ways to compensates for distortions, non-uniformities or anomalies in the lines, dots or distributions in the test strip.

9. The device of claim 1, in which the image processing software correlates distortions, non-uniformities or anomalies in the lines or distributions in the test strip to variables, such as batch numbers, and environmental factors.

10. The device of claim 7, in which digital filters are applied to the captured image signal of the test strip.

11. The device of claim 1, in which connected devices all have access to a cloud-based model or library.

12. The device of claim 1, in which four LEDs of different colors, selected from the group Red, Green, Blue and White or UV, are used to illuminate the test strip.

13. The device of claim 1, in which the imaging sensor images an amount of the surface of the test strip selected from the group: substantially the entire surface of the test strip; or at least 90% of the entire surface of the test strip; at least 80% of the entire surface of the test strip; at least 70% of the entire surface of the test strip; at least 60% of the entire surface of the test strip.

14. The device of claim 1, in which the image processing software includes a distortion compensation algorithm taking into account one or more of: the lens parameters, the location of the LEDs in relation to the lens and the position of the test strip in relation to the cartridge.

15. The device of claim 1, in which the image processing software includes a distortion compensation algorithm that further takes into account the results of a calibration obtained by imaging a color and/or white charts.

16. The device of claim 1, in which the lens simultaneously or near simultaneously images a substantial part of the 2D surface of two or more adjacent immunoassay test strips.

17-25. (canceled)

26. The device of claim 1, in which the device images a uniformly colored test region, such as a white region, to measure and compensate for lens vignetting.

27. The device of claim 1, including two or more camera modules, each camera module including an imaging sensor and a lens that images at least a part of the 2D surface of the immunoassay test strip onto the imaging sensor, and in which the two or more camera modules have different specifications.

28. The device of claim 27, in which one or more of the camera modules includes a commodity non-custom lens and commodity CMOS sensor that inherently introduce distortions when creating an image of the substantial part of the 2D surface.

29. The device of claim 27, in which the camera modules have different specifications, selected from the group: a different FOV, a different resolution, a different pixel size, a different sensitivity, a different filter or filters.

30. The device of claim 27, in which a first camera module includes a color imaging sensor and a second camera module includes a black and white imaging sensor.

31. The device of claim 27, in which each camera module is configured to image a specific portion of the test strip or to image a specific portion of a cartridge carrying the test strip.

32. The device of claim 27, in which each camera module is configured to image a specific type of test strip.

33. The device of claim 27, in which one camera module includes a global shutter to read out fluorescent based lateral flow test.

34. The device of claim 1, in which the lens is a wide angle lens that is configured to automatically self-calibrate in the color space by imaging a color and/or white chart, in which the color and/or white charts are built into part of the device and are also present on a dedicated cartridge or cartridge carrying adaptor that is slid into the device.

35. The device of claim 34 in which the imaging software uses the data from the calibration process to compensate for temperature dependent drift in the color mapping of the imaging sensor.

36. The device of claim 34, in which the imaging software uses the data from the calibration process to ensure that data from multiple different devices analyse consistently.

37. The device of claim 34, in which the calibration curve associated with imaging a specific test strip held in a cartridge is written to a memory or record on or associated with that cartridge, including a QR code engraved or printed on the cartridge or an RFID tag or other memory on the cartridge or adaptor.

38. The device of claim 34, in which imaging the white chart from a dedicated calibration cartridge slid into the device determines the following calibration parameters: exposition value and/or vignette compensation parameters.

39. The device of claim 34, in which imaging the color charts from a dedicated calibration cartridge slid into the device determines the following calibration parameters: image scale factor, a color correction matrix and/or the image sensor's displacement.

40. The device of claim 34, in which calibration parameters are transmitted and stored into a cloud for subsequent analysis of test strips.

41. The device of claim 1 that is configured to receive and also analyse cartridges for both lateral flow immunoassay test strips and also micro-array immunoassays.

42. The device of claim 1 that includes a UV light source to illuminate the immunoassay test strip.

43. The device of claim 42, in which a UV filter is included in front of the wide angle lens.

44. The device of claim 1, in which the device includes a short range, secure communications link or interface that enables the device to exchange data with an application running on a smartphone or other wireless device, and the smartphone or other wireless connected device then connects over a secure link via the internet or a cellular network to a remote, cloud-based server.

45. The device of claim 1, in which the device accepts different types of cartridge carrying adaptor that are configured to hold any commercially available test, such as electrochemical test, reflectance photometry based test, rapid lateral flow assay, test strip or dipsticks.

46. The device of claim 45, in which a cartridge carrying adaptor includes various sets of actuators or sensors in order to perform the different tests.

47. The device of claim 45, in which a cartridge carrying adaptor includes a temperature control system for controlling the temperature in the cartridge when a test is being performed.

48. The device of claim 45, in which a cartridge carrying adaptor includes a mechanical fixture arranged to move a dipstick test along the cartridge carrying adaptor, such that portions of the dipstick test are read out sequentially by the device.

49. The device of claim 44, in which the results of the different types of test are transmitted, analysed and combined on the server.

50. The device of claim 1, in which the device includes a cartridge, the cartridge including or associated with a read/write memory to which is written, in normal operation by the device, the kinetic data and the calibration curve obtained when the device images a test strip mounted on or associated with that cartridge.

51. The cartridge of claim 50, in which the calibration curve is a color calibration curve.

52. The cartridge of claim 50, in which the cartridge is configured by shape and size to slide into an aperture in the device.

53. The cartridge of claim 50, in which the read/write memory is an integral part of or attached to the cartridge.

54. The cartridge of claim 50, in which the read/write memory is an integral part of or attached to an adaptor for the cartridge,

55. The cartridge of claim 50, in which the cartridge memory has written to it one or more of the following: the test type, expiration date, test date, test time, test calibration date, calibration curve for this batch or lot number, test manufacturer, test manufacturer address, test manufacturer contact information.

56. The cartridge of claim 50, in which the calibration curve is updated when a subsequent test strip result is obtained.

57. The cartridge of claim 50, in which the calibration curve is updated from the results stored on the diagnostic device or on a cloud-based library that is accessible by the diagnostic device.

58. (canceled)

59. The device of claim 1, in which the device includes a cartridge, the cartridge including a secure memory chip or device and a communications chip or device, the secure memory storing a unique identification code or crypto-key or number and the cartridge configured to undertake a handshake or other protocol in which the unique identification code or crypto-key or number is checked and verified as authentic or otherwise genuine.

60. The device of claim 1 that includes multiple LEDs of different colors used to read the test strip, in which the device can be programmed to select only a set of LEDs to be turned on, in order to image a specific region of interest of the 2D surface of the immunoassay test strip onto the color imaging sensor.

61. The device of claim 60, in which a set of LEDs located above the lens is turned off, and a set of LEDs located below the lens is turned on, in order to image the bottom region of the test strip.

62. The device of claim 60, in which a set of LEDs located below the lens is turned off, and a set of LEDs located above the lens is turned on, in order to image the top region of the test strip.

63. The device of claim 60, in which the results obtained from imaging the bottom region and top region of the test strip are combined.

64. The device of claim 1, in which the device is configured to capture the progress or change or rate of change of the lateral flow test strip as a function of time (‘kinetic data’).

65. The device of claim 64, in which the device includes an image processing software that identifies as a function of time patterns in the test lines and/or dots or other distributions in the test strip and compensates for distortions, non-uniformities or anomalies in the lines and/or dots or other distributions in the test strip.

66. The device of claim 64, in which kinetic data associated with imaging a specific test strip held in a cartridge is written to a memory or record on or associated with that cartridge, including a QR code engraved or printed on the cartridge or an RFID tag or other memory on the cartridge or adaptor.

67. The device of claim 64, in which imaging software uses the kinetic data to obtain or measure the lateral flow test result.

68. The device of claim 64, in which by comparing the stored kinetic data, the device is able to forecast what the rate of change of the test strip should look like.

69. The device of claim 64, in which by analysing the progress or rate of change of the test strip as a function of time, the device is able to detect errors early.

70. The device of claim 64, in which images of the substantial part of the 2D surface of the lateral flow test strip are captured at predefined intervals, such as every n-seconds.

71. The device of claim 64, in which images of the substantial part of the 2D surface of the lateral flow test strip are captured at predefined intervals and for a predefined duration, each being determined by the specific test being undertaken.

72. The device of claim 64, in which predefined intervals and predefined duration are included in the kinetic data stored on a cartridge.

73. The device of claim 64, in which the device provides the progress or change, or rate of change of the lateral flow test strip as a function of time to a data analysis system that uses that kinetic data to improve the coefficients of variation of test results.

74. The device of claim 64, in which the device monitors whether or not the lateral flow test strip changes during a predefined initial period, such as the first n seconds, in a way that is consistent with the test operating correctly; and if no such changes are detected, then the device generates an alert.

75. The device of claim 64, in which the kinetic data is compared to a statistical library or model of kinetic data for both successful and failed tests to determine the likelihood of the test being successful, and, if the possible or likely causes of any unsuccessful test.

76. The device of claim 1 that is configured to automatically run a diagnostic test result following a one-step user interaction with the device.

77. The device of claim 76, in which the diagnostic test result is automatically performed once a dedicated cartridge or cartridge carrying adaptor is slid and inserted in place inside the portable diagnostic device.

78. The device of claim 77, in which when a cartridge is already inserted in the portable diagnostic device, the diagnostic test result is automatically performed after tapping the portable diagnostic device.

79. The device of claim 78, in which when a cartridge is already inserted in the portable diagnostic device, the diagnostic test result is automatically performed when the diagnostic device is selected on a connected application.
